(19) United States
(12) Patent Application Publication
(10) Pub. No.: US 2023/0226230 A1
(43) Pub. Date: Jul. 20, 2023

(54) THIOPURINE-BASED COMPOUND, COMPOSITION, METHOD OF PREPARATION AND APPLICATIONS

(71) Applicant: SEECURE TAIWAN CO., LTD., Kaohsiung (TW)

(72) Inventors: Wei-Chung CHANG, Kaohsiung (TW); David J. YANG, Kaohsiung (TW); Min-Ching CHUNG, Kaohsiung (TW); Chi-Shiang KE, Kaohsiung (TW); Tsung-Tien KUO, Kaohsiung (TW)

(73) Assignee: SEECURE TAIWAN CO., LTD., Kaohsiung (TW)

(21) Appl. No.: 17/802,190

(22) PCT Filed: Feb. 26, 2021
(86) PCT No.: PCT/CN2021/078187

§ 371 Date: Aug. 25, 2022

(51) Int. Cl. A61K 51/04; C07D 473/38; A61P 35/00

(52) U.S. Cl. CPC A61K 51/0497; C07D 473/38; A61P 35/00

(57) ABSTRACT

A compound for measurement of thiopurine pathway directed systems imaging and therapy including a chelator and a thiopurine ligand is provided. A method of synthesizing the compound is also provided, and the compound may be further prepared in pharmaceutical formulations or kits for therapy or molecular imaging.

### TECHNICAL FIELD

The present invention relates to a compound for theranostic (diagnosis and therapeutic) of functional cellular deficiencies with genetic etiology, proteasome expression differences, and chromosomal abnormalities; a method of synthesizing the same; an imaging method for surrogate end-points in clinical trials, and a treatment method for optimal response outcome using the same.

### DESCRIPTION OF RELATED ART

Healthcare management relies on computed tomography (CT), magnetic resonance imaging (MRI), x-ray or ultrasound. These modalities provide morphological (size, shape) and anatomical information, but not with cellular target information. Thus, assessment of the effectiveness of therapeutic response is not at optimal. The treatment endpoints rely almost exclusively on the analysis of biopsies by molecular and histopathological methods which are invasive and with sampling errors [1-3].

Positron emission tomography (PET) and single photon emission computed tomography (SPECT) radiopharmaceuticals are able to image, map, and measure target site activities. PET and SPECT agents are considered as molecular imaging agents as well as micro-dosing agents because they do not induce detectable pharmacologic effects. Molecular and cellular image-guided customized therapy made it possible to assess the efficacy of tumor therapy by measuring changes in proteasome and proliferative activity. The success of this endeavor to noninvasively detect the degrees of tumor proteasomes and proliferation would enable physicians to select additional or alternative treatment regimens for optimal respond rate and reduce the costs by avoiding unnecessary treatments.

Sequential measurements of proteasome concentration and DNA proliferative activity by a molecular imaging agent would allow measurement of tumor targets on a whole-body image as well as monitor the effects of therapy. In addition, molecular DNA agents would differentiate inflammation or scar tissue versus tumor recurrence. Moreover, molecular DNA agents provide opportunities to predict the response to chemotherapy and radiation therapy which may discontinue ineffective treatment in the earlier phase and be beneficial to patients.

Several efforts have been made to assess tumor proliferative activity. It has been reported that 2′-fluorodeoxyglucose ([¹⁸F]FDG) uptake is an indicator of tumor proliferative activity [4-6]. Higashi et al. have shown that [¹⁸F]FDG uptake is strongly related to the number of viable cells [7]. Another approach was to use radiolabeled amino acid as a tumor cell proliferative marker [8-12]. However, the structures of these agents are not purine or pyrimidine-based which are essential building blocks of DNA/RNA. Several radiolabeled pyrimidine and purine have been developed. They were used as probes for imaging herpes virus type one thymidine kinase (HSV1-tk) expression and other reporter genes [13-27]. The difficulty of these probes in imaging is that HSV1-tk enzyme expression depends on HSV1-tk gene transduction with an adenoviral type vector. The level of HSV1-tk enzyme expression is likely to be altered in different transduced cells and tissue, thus, the application of HSV1-tk probe is limited.

To overcome the efficiency of gene therapy using HSV1-tk probes, several pyrimidine and purine nucleosides/nucleotides were synthesized and attempted to be incorporated into DNA/RNA [23-29]. For instance, 3′-deoxy-3′-¹⁸F-fluorothymidine (¹⁸F-FLT) is a tracer which images cellular proliferation by entering the salvage pathway of DNA synthesis. Although FLT uptake correlated significantly with proliferative activity in certain type of cancer, yet the tumor uptake was low [23,24]. The application of FLT for evaluation of primary or recurrent low-grade tumor or post-treatment outcome measurement is limited.

Overall, radionuclide imaging modalities could assess (1) cellular targets at low cost (2) treatment response more rapidly, (3) differential diagnosis, (4) the prediction of therapeutic response, and (5) better dosimetry for internal radiotherapy. For clinical use in patients the choice should be determined not only by the biological behavior of radiopharmaceuticals but also by its ease of preparation, as well as by the logistics of imaging in real time with new software to manipulate the information gather from the patient so that better treatment planning could be designed. To assess cellular proliferative activity, chelator-based purine analogue was selected due to its involvement in both m-RNA and adenosine 5′-triphosphate (ATP) and guanosine 5′-triphosphate (GTP) pathways. Chelator-based purine imaging agents provide opportunities to characterize tumor aggressiveness, grade of the cancer, purine pathway-directed systems therapy and allow clinicians to select the patient for optimal outcome by using customized treatment. In addition, the molecule may be incorporated with therapeutic radionuclide for internal radionuclide therapy.

### SUMMARY OF THE INVENTION

What would have been obvious to a person of ordinary skill in the prior art include: (1) the sophistication of the ¹⁸F-radiochemistry in imaging; (2) the treatment outcome was not optimal due to lack of theranostic concept; (3) not able to transform a biomarker to an inverse agonist. An inverse agonist may avoid adverse event and provide correlative analysis between a biomarker and post-therapeutic response; and (4) difficulty in differential diagnosis and responsive prediction due to either lack of DNA target delineation or use different molecules in imaging and treatment.

The non-obviousness in the art includes: (1) The chelation-conjugate technology platform allows the selection of patients for optimal treatment dosage response due to its ability to enhance sensitivity and specificity of the drug; (2) The chelation-conjugate technology platform could transform a simple kit-based imaging drug to a predictive therapeutic drug, which is a theranostic concept for customized medication; (3) The chelation-conjugate technology platform could transform a biomarker (agonist) to an inverse agonist; and (4) The SC-06 thiopurine compounds, chelation-conjugate technology-driven product, could detect tumors regardless fast growing or slow growing tumors due to its ability involving in DNA proliferation. Understanding the different type of tumors proliferative activities could aid in the selection of patients for optimal therapy. The chelator-based purine conjugates were developed to assess efficacy of tumor therapy by measuring changes in proteasome conversions and proliferative activity.

The invention provides a compound for quantifying purine pathway-directed systems, which has the following formula:

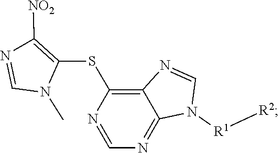

wherein R¹ is an alkyl group containing 2 to 7 carbon atoms, and one of the carbon atoms is optionally substituted by a hydroxy group; R² is a chelator with a nitrogen containing tetraazacyclic ring.

In an embodiment of the invention, the chelator is cyclam, cyclen, cyclam-carboxylic acid, or cyclen-carboxylic acid.

In an embodiment of the invention, the chelator is chelating a metal ion.

In an embodiment of the invention, the metal ion is a radionuclide, a non-radioactive metal, or a combination thereof.

In an embodiment of the invention, the radionuclide is ^{99m}Tc, ^{67,68}Ga, ^{60,61,62,64,67}Cu, ¹¹¹In, ¹⁶⁶Ho, ^{186,188}Re, ⁹⁰Y, ¹⁷⁷Lu, ²²³Ra, ²²⁵Ac, and ⁸⁹Zr, ^{117m}Sn, ¹⁵³Sm, ⁸⁹Sr, ⁵⁹Fe, ²¹²Bi, ²¹¹At, ⁴⁵Ti, or a combination thereof.

In an embodiment of the invention, the non-radioactive metal is Tc, Sn, Cu, In, Tl, Ga, As, Re, Ho, Y, Sm, Se, Sr, Gd, Bi, Fe, Mn, Lu, Co, Pt, Ca, Rh, Eu, Tb, or a combination thereof.

In an embodiment of the invention, the compound has one of the following formulas:

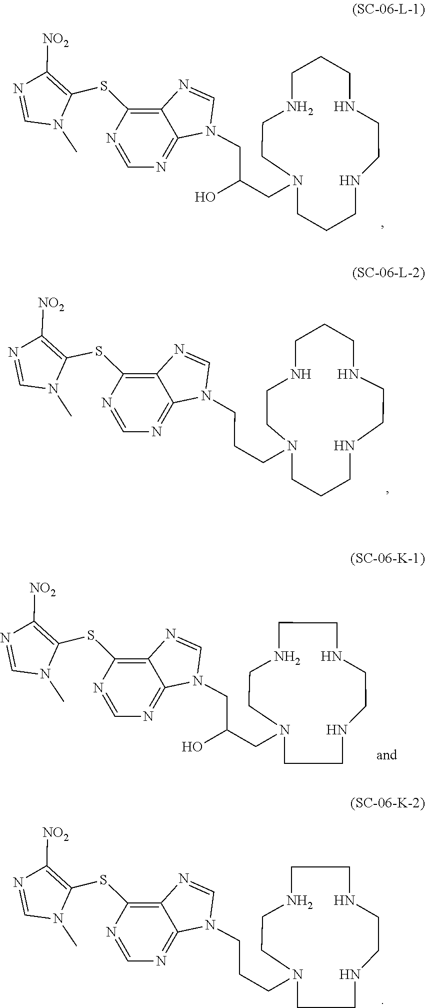

The invention also provides a kit including the compound described above and an instrument for administering the pharmaceutical compound.

The invention further provides a method of synthesizing the compound described above. The method comprise:

reacting a compound represented by Chemical Formula 1 with a compound represented by Chemical Formula 2

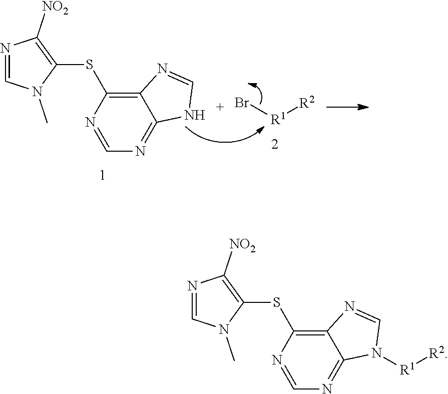

The invention further provides a method of scanning tumors, which comprise administering to an animal containing tumors an imaging amount of the compound described above, and imaging the bones with imaging technology for detecting cancer, autoimmune disorder (multiple sclerosis, myasthenia gravis, rheumatoid arthritis, systemic lupus erythematosus/Lupus nephritis, inflammatory bowel disease, ulcerative colitis, idiopathic pulmonary fibrosis, hepatitis), bone marrow disease, atherosclerosis.

In an embodiment of the invention, the image technology is CT, MRI, PET and/or SPECT.

In an embodiment of the invention, the imaging amount is defined as a kit.

The invention further provides a treatment method for cancer, anti-metabolite, anti-proliferation (S/G1), autoimmune disorder (multiple sclerosis, myasthenia gravis, rheumatoid arthritis, systemic lupus erythematosus/Lupus nephritis, inflammatory bowel disease, ulcerative colitis, idiopathic pulmonary fibrosis, hepatitis), bone marrow disease, comprising administration of the compound described above.

In an embodiment of the invention, a spacer with a hydroxy group is incorporated in the molecule (SC-06-L-1).

The technology platform may exploit conjugating antagonists (drug) and agonists (biomarker) and seeing their effects in various forms of diseases.

Also, the compound may be further prepared in pharmaceutical formulations and kits using chemical procedures known to skilled artisans. In addition, the method of synthesizing the compound is also provided, and the synthesis method may obviate the need of adding protecting groups to the thiopurine ligand and increase process efficiency and purify of the final product.

The present invention provides the compound of theranostic (diagnosis and therapeutic) proteasomes for thiopurine methyltransferase (TPMT), hypoxanthine guanine phosphoribosyl transferase (HPRT) and xanthine oxidase (XO) for functional deficiencies with a genetic etiology, protein expression differences, and chromosomal abnormalities.

### BRIEF DESCRIPTION OF THE DRAWINGS

To make the aforementioned more comprehensible, several embodiments accompanied with drawings are described in detail as follows. The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1. Scheme of values for a radiomic theranostic agent: staging, re-staging, accelerate response prediction and improve efficacy/cost ratio.

FIG. 2. Molecule structures of purine derivatives.

FIG. 3. Molecule structures of thiopurine and guanine derivatives.

FIG. 4. Technology platform of SC-06 Analogues.

FIG. 5. SC-06 analogues for theranostic of CD 28, G-protein, and DNA proliferation.

FIG. 6. Putative Mechanism of Action-1: SC-06 activates CD-28 and inhibits Rac-1 (left), and cross-talks to receptor kinase pathways (right) for differential responsiveness outcome in cancer.

FIG. 7. Putative Mechanism of Action-2: Involvement of SC-06-L1 in DNA/RNA incorporation in thiopurine pathway-directed systems (MP: mercaptopurine; TG: thioguanine).

FIG. 8. SC-06 compounds measure enzymatic (XO, HPRT, TPMT) conversions in thiopurine pathway-directed systems.

FIG. 9. Synthetic scheme of SC-06-L-1.

FIG. 10. ¹H NMR of SC-06-L-1 (B082-1).

FIG. 11. ¹³C NMR of SC-06-L-1 (B082-1).

FIG. 12. Mass of SC-06-L-1 (B082-1).

FIG. 13. HPLC of SC-06-L-1 (B082-1).

FIG. 14. Synthetic scheme of SC-06-L-2.

FIG. 15. ¹H NMR of SC-06-L-2 (B040-2).

FIG. 16. ¹³C NMR of SC-06-L-2 (B040-2).

FIG. 17. Mass of SC-06-L-2.

FIG. 18. HPLC of SC-06-L-2.

FIG. 19. Synthetic scheme of SC-06-K-1.

FIG. 20. ¹H NMR of SC-06-K-1.

FIG. 21. ¹³C NMR of SC-06-K-1.

FIG. 22. HPLC of SC-06-K-1.

FIG. 23. Mass of SC-06-K-1.

FIG. 24. Synthetic scheme of SC-06-K-2.

FIG. 25. ¹H NMR of SC-06-K-2 (A177).

FIG. 26. ¹³C NMR of SC-06-K-2 (A177).

FIG. 27. Mass of SC-06-K-2 (A177).

FIG. 28. HPLC of SC-06-K-2.

FIG. 29. Instant radio-TLC analysis of ^{99m}Tc-SC-06 analogues (saline as an eluant) showed Rf=0.1 using cut-and-count technique. The radiochemical purity of Compound ^{99m}Tc-SC-06-K synthesized in the invention was greater than 90%.

FIG. 30. Instant radio-TLC analysis of ^{99m}Tc-SC-06 analogues (saline as an eluant) showed Rf=0.1 using cut-and-count technique. The radiochemical purity of Compound ^{99m}Tc-SC-06-L synthesized in the invention was greater than 85%.

FIG. 31. In Vitro cell/media ratios (volume of distribution) in breast cancer cells showed that ^{99m}Tc-SC-06-L1 had high uptake in time-dependent patterns which had potential for imaging breast tumors.

FIG. 32. In Vitro cell/media ratios (volume of distribution) in ovarian cancer cells showed that ^{99m}Tc-SC-06 analogues had high uptake in time-dependent patterns which had potential for imaging ovarian tumors.

FIG. 33. In Vitro MTT cytotoxicity assays of SC-06-K analogues against various human lymphoma cells indicating SC-06-K-1 inhibited cancer cells in a dose-dependent manner.

FIG. 34. In Vitro MTT cytotoxicity assays of SC-06-K-1 against various human diffuse large B-cell lymphoma cells indicating SC-06-K-1 inhibited cancer cells in a dose-dependent manner.

FIG. 35. ^{99m}Tc-SC-06-L1 and L2 images at 1 hr revealed high tumor uptake in an athymic nude mouse-bearing human ovarian tumors (OVCA-3 and TOV-2d) whereas ¹⁸F-FDG had poor uptake.

FIG. 36. Compare to ¹⁸F-FDG, SC-06 compounds showed significant increased uptake in tumors. ^{99m}Tc-SC-06-L1 demonstrated the superior pharmacokinetic properties and high contrast visualization in ovarian tumors.

FIG. 37. Biodistribution of ^{99m}Tc-SC-06-L1 in nude mice-bearing human ovarian tumors showed OCAR-3 tumor had more uptake than TOV-112d.

FIG. 38. ^{99m}Tc-SC-06-L1 and ^{99m}Tc-SC-06-K1 showed high uptake in nude mice bearing MDA-MB231 and MCF tumors compared to ¹⁸F-FDG.

FIG. 39. Compare to ¹⁸F-FDG, SC-06 compounds showed significant increased uptake in tumors. ^{99m}Tc-SC-06-L1 demonstrated the superior pharmacokinetic properties and high contrast visualization in breast tumors.

FIG. 40. Two nude mice were inoculated with two types of breast tumors (MDA-MB-231 and MCF-7) and administered ¹⁸F-FDG. Tumor/muscle count density ratios at 0.5 hrs were 0.25 (MDA-MB-231) and 0.28 (MCF-7), respectively.

FIG. 41. Two nude mice were inoculated with two types of breast tumors (MDA-MB-231 and MCF-7) and administered ^{99m}Tc-SC-06-L1. Tumor/muscle count density ratios at 1-6 hrs were 2.0-3.0 (MDA-MB-231) and 2.1-3.2 (MCF-7), respectively.

FIG. 42. Two nude mice were inoculated with two types of breast tumors (MDA-MB-231 and MCF-7) and administered ^{99m}Tc-SC-06-K1. Tumor/muscle count density ratios at 1-6 hrs were 1.2-2.4 (MDA-MB-231) and 1.6-2.2 (MCF-7), respectively.

FIG. 43. Nude mice bearing MDA-MB231 (right thigh) and MCF (left thigh) tumors were administered ^{99m}Tc-SC-06-L1 and ^{99m}Tc-SC-06-K1 (120 uCi/100 uL per mouse, n=2/compound, iv). The whole-body images were collected by an eZ-Scope (Anzai Medical, Japan) at 1-6 hrs. Tumor/muscle count density ratios by ^{99m}Tc-SC-06-L1 and ^{99m}Tc-SC-06-k1 in both MDA-MB231 and MCF-7 tumors ranged 2.0-3.2 and 1.2-2.4, respectively.

### DESCRIPTION OF THE EMBODIMENTS

References are in detail to the present preferred embodiments of the invention, examples of which are illustrated in the accompanying drawings. The same reference numbers are used in the drawings and the description to refer to the same or like parts.

Accessible, Available and Affordable of Generator-Produced Isotopes for Imaging

Cyclotron-produced tracers are constrained by the availability of local cyclotron, alternatively, radionuclide generator systems that can be produced in a well-controlled facility and have a long history of successful clinical application. A generator uses a parent-daughter nuclide pair wherein a relatively long-lived parent isotope decays to a short-lived daughter isotope that is used for imaging. The parent isotope, which is produced at a cyclotron facility, can be shipped to a clinical site and from which the daughter isotope may be eluted on site for clinical use. For instance, ^{99m}Tc-(6 hrs half-life) and ⁶⁸Ga-based (68-minute half-life) are with significant commercial potential because the isotopes can be produced from the generators on site and are convenient alternative to cyclotron-produced isotopes, such as ¹⁸F- or ¹²⁴I-. The uniqueness of the chelator-conjugate kit is that the instant kit can be easily trapped with radioisotope ^{99m}Tc-(SPECT) and ⁶⁸Ga (PET) known as “click chemistry” with high radiochemical purity and stability for imaging, or be used as a therapeutic agent by entrapping the therapeutic metal. Thus, the chelation technology platform is the foundation to develop theranostic molecules.

Significance of Imaging Cell Proliferation in Cancers

Contemporary CT, MRI and ultrasound imaging modalities could detect tumor volumetric and morphological changes but not accurately assess the extent of tumor proteasome and proliferation changes. Pathologic examination is needed to confirm the diagnosis of proliferation. Because of the continuing need for diagnostic and therapeutic tests, efforts have been made to develop more efficient and accurate noninvasive methods to detect the extent of tumor proliferation, which correlates with tumor behavior.

Currently, to eradicate solid tumors is by repeated exposures to high doses of chemotherapy. The success of such high-dose therapy is often limited by the myelosuppressive and toxic effects of these drugs on bone marrow cells and by the intrinsic resistance of the cancer cells to chemotherapy. The increased level of multi-drug resistance (MDR) expression and resistance to chemotherapy are associated with proteasomes and DNA proliferative activity [30-31]. Tumor proliferation rates have been found to be directly related to survival and prognosis.

Though PET [¹⁸F]Fluorodeoxyglucose (FDG), a clinic gold standard in imaging, was concordant with the findings of CT and MRI in diagnosing various tumors, FDG also has a drawback in its use for personalized cancer treatment due to shortage of downstream specific cellular pathways. A significant amount (>95%) of FDG was concentrated in mitochondria fraction and this resulted in an apparent false-positive lesion between inflammation/infection and tumor recurrence [32]. Thus, FDG is a sensitive but not specific biomarker for the prediction of therapeutic response. [¹⁸F]Fluoro-L-Thymidine (FLT) was developed for imaging changes in genes, RNA, or DNA in primary and recurrent tumors, however, the uptake was low in addition to its complex chemistry [23,24,33]. In the present invention, thiopurine-conjugate kit was applied for the measurement of DNA/RNA proliferation activity by imaging. The imaging approach that advances understanding of thiopurine pathway-directed systems with potential to apply thiopurine-conjugate for therapeutic avenues to modulate DNA proliferation in cancer.

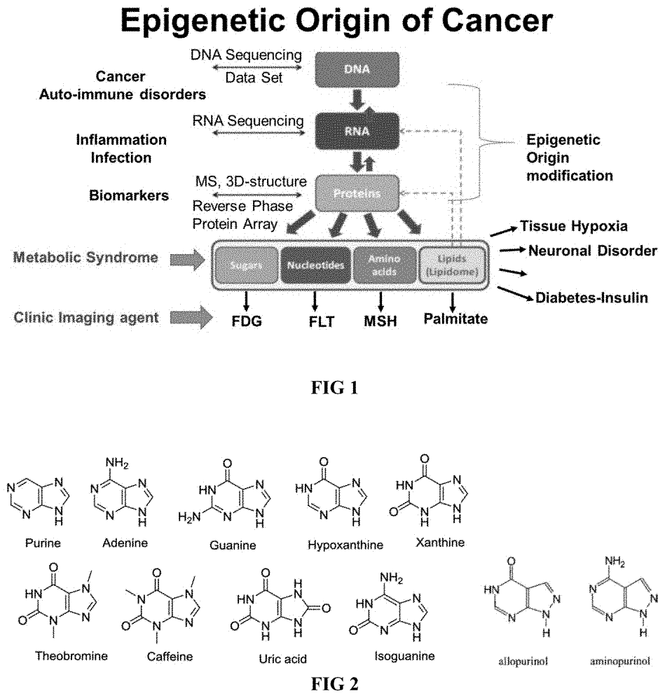

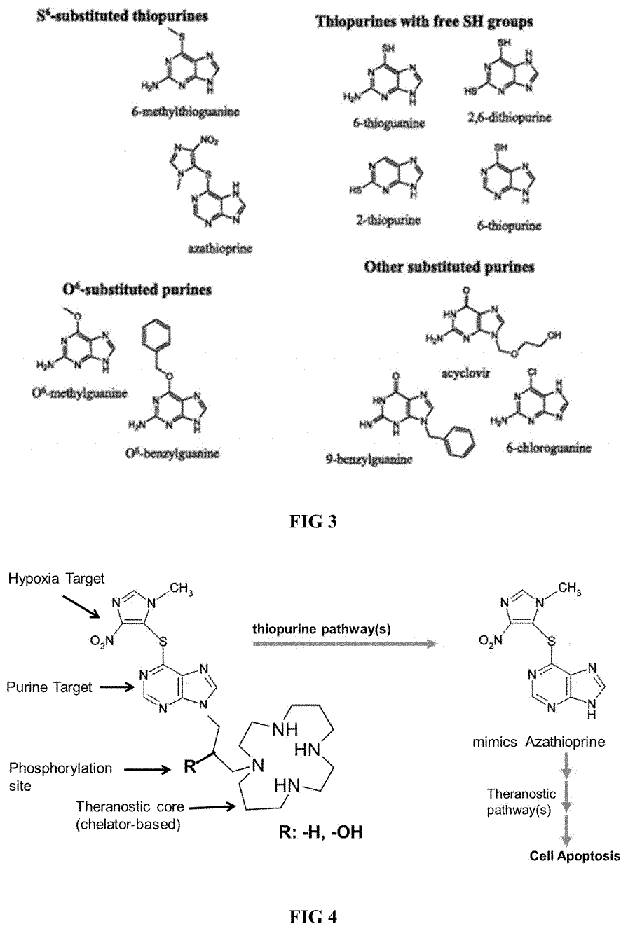

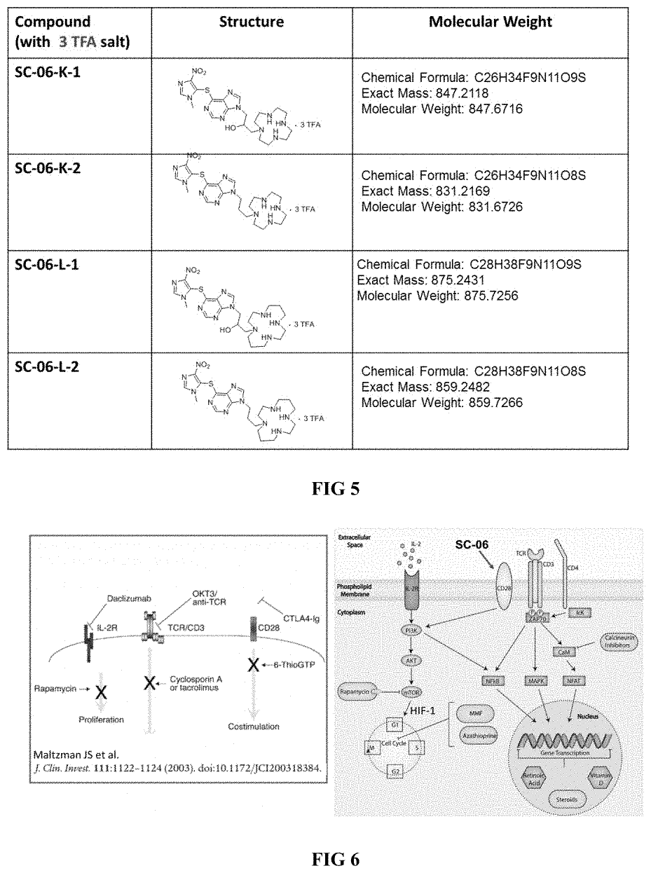

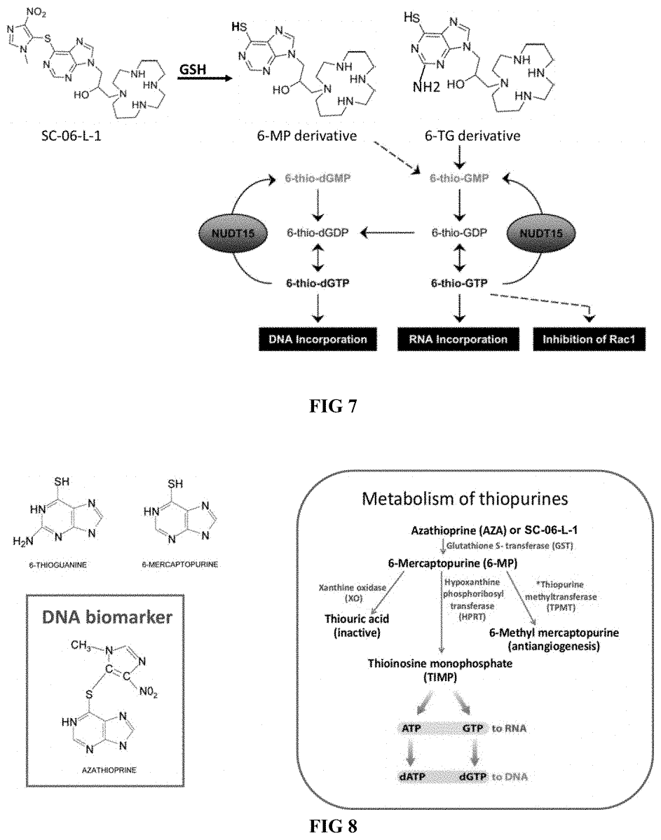

DNA genes regulate RNA and RNA transcribes various proteins in cellular pathways. Proteomic activities are real-time and dynamic over-expressions in various tumors. FIG. 1 shows scheme of values for a radiomic theranostic agent: staging, re-staging, accelerate response prediction and improve efficacy/cost ratio. Thus, it is amenable to develop a radiopharmaceutical to measure proteasome and DNA activities which may provide therapeutic prediction beyond FDG and FLT. Purine structures are either adenine (FIG. 2), thiopurine or guanine (FIG. 3) derivatives. The technology platform was to manufacture chelator-thiopurine conjugate that mimics azathioprine pathway-activated systems (FIG. 4). The chelators selected for conjugation are cyclam and cyclen (FIG. 5). For instance, cyclam-azathioprine (SC-06-L-1) mimics azathioprine pathway, binds to CTLA, activates CD-28 inhibits Rac-1 signaling. FIG. 6 shows the putative mechanism of action-1: SC-06 activates CD-28 and inhibits Rac-1 (left), and cross-talks to receptor kinase pathways (right) for differential responsiveness outcome in cancer. SC-06-L-1 also cross-talks to receptor kinase pathways for differential responsiveness outcome in cancer. FIG. 7 shows the putative mechanism of action-2: Involvement of SC-06-L1 in DNA/RNA incorporation in thiopurine pathway-directed systems (MP: mercaptopurine; TG: thioguanine). SC-06-L-1 maps thiopurine pathway-directed systems in DNA/RNA incorporation. Radiolabeled SC-06 compounds measure enzymatic (XO, HPRT, TPMT) conversions in thiopurine pathway-directed systems (FIG. 8). In summary, radiolabeled chelator-thiopurine conjugate may allow precisely measurement of tumor targets on a whole-body image which may monitor and predict response to chemotherapy and radiation therapy. Ultimately, chelator-purine probes may discontinue ineffective treatment in the earlier phase and be beneficial to patients.

Design of Chelator-Purine as a Probe for Purine Pathway-Directed Systems Theranostics

Nucleotides such as adenosine 5′-triphosphate (ATP) and guanosine 5′-triphosphate (GTP) are as building blocks for DNA and RNA. They are crucial for providing cellular energy and intracellular signaling. Higher intracellular purine levels as well as enzymes are upregulated via biosynthetic pathway under tumor cell proliferation upregulating. Purines can also be incorporated into more complex biomolecules and serve as cofactors such as nicotinamide adenine dinucleotide (NAD) and coenzyme to promote cell survival and proliferation.

Thiopurines are chemically more reactive than the normal DNA purines. For instance, azathioprine has been used as an immunosuppressant with T- and B-cell proliferation inhibition for the treatment of hematologic malignancies, rheumatologic diseases, solid organ transplantation, and inflammatory bowel disease. Azathioprine is metabolized through reduction by glutathione and then enzymatically converted by hepatic xanthine oxidase (XO) to its active metabolite 6-mercaptopurine (6-MP). 6-MP is further metabolized by hypoxanthine-guanine phosphoribosyl transferase (HPRT) into 6-thioguanosine-5′-phosphate (6-thio-GMP) and 6-thioinosine monophosphate (6-thio-IMP), both inhibit nucleotide conversions and de novo purine synthesis. This leads to inhibition of DNA, RNA, and protein synthesis. Ultimately, azathioprine can then become incorporated into replicating DNA and can also block the de novo pathway of purine synthesis. Thiopurine methyltransferase (TPMT), xanthine oxidase (XO) and HPRT are thought to contribute to its relative specificity to lymphocytes [34-36]. Radiolabeled chelator-azathioprine conjugate would be the choice to assess cellular proliferative activity because it is involved in m-RNA and enzymatic (XO, HPRT, TPMT) interconversion to guanine in DNA pathways and serve as a surrogate biomarker (FIG. 8). In addition, the choice of molecules should be determined not only by the biological behavior of radiopharmaceuticals but also by its ease of preparation, as well as by the logistics of imaging in real time with software to quantify the changes in tumors gather from the patient so that better treatment planning may be designed.

In some embodiments, the chelator may be a nitrogen containing tetraazacyclic ring, for example. Specifically, the nitrogen containing tetraazacyclic ring may be a cyclam, a cyclen, a cyclam-carboxylic acid, or a cyclen-carboxylic acid, for example, but the invention is not limited thereto.

In some embodiments, the purine ligand may be a thiopurine ligand or a nitroimidazole thiopurine ligand, or a guanine ligand for example. In some embodiments, the purine ligand may include azathioprine, for example. However, the invention is not limited thereto. In some embodiments, the purine ligand has a spacer hydroxy group which will be described in detail below.

In some embodiments, the compound further includes a metal ion. Specifically, the metal ion may be a radionuclide, a non-radioactive metal, or a combination thereof, for example.

In some embodiments, the radionuclide may be ^{99m}Tc, ^{67,68}Ga, ^{60,61,62,64,67}Cu, ¹¹¹In, ¹⁶⁶Ho, ^{186,188}Re, ⁹⁰Y, ¹⁷⁷Lu, ²²³Ra, ²²⁵Ac, and ⁸⁹Zr, ^{117m}Sn, ¹⁵³Sm, ⁸⁹Sr, ⁵⁹Fe, ²¹²Bi, ²¹¹At, ⁴⁵Ti, or a combination thereof, for example. In some other embodiments, the non-radioactive metal may be a technetium ion (Tc), a stannous ion (Sn), a copper ion (Cu), an indium ion (In), a thallium ion (Tl), a gallium ion (Ga), an arsenic ion (As), a rhenium ion (Re), a holmium ion (Ho), a yttrium ion (Y), a samarium ion (Sm), a selenium ion (Se), a strontium ion (Sr), a gadolinium ion (Gd), a bismuth ion (Bi), an iron ion (Fe), a manganese ion (Mn), a lutecium ion (Lu), a cobalt ion (Co), a platinum ion (Pt), a calcium ion (Ca), a rhodium ion (Rh), an europium ion (Eu), and a terbium ion (Tb), or a combination thereof, for example. However, the invention is not limited thereto. In one specific embodiment of the invention, the compound may be a ^{99m}Tc-cyclam-azathioprine analogue. In another specific embodiment of the invention, the compound may be a ^{99m}Tc-cyclen-azathioprine analogue.

It should be mentioned that the compound of the present invention may be used to identify the proteasome over-expression through cell surface nucleoside transporter. Radiolabeled purine ligand is not only able to quantify cell proliferation to stage and re-stage of the cancer, but also able to select the patients for optimal response to therapy as well as to discontinue the treatment when resistance occurs. In other words, due to the structure of the compound, the compound may quantify drug to proteasome (XO, HPRT, TPMT) binding pocket by an active transport strategy, thereby overcoming the drug resistance.

The present invention further provides a method of synthesizing the compound. The steps of the synthesis method are described in detail below, but the invention is not limited thereto.

In some embodiments, the di-bromohydrin is conjugated to a tetracyclic ring first, for example. In other words, the mono bromohydrin is attached to a purine ligand. In some embodiments, the purine ligand may be an antagonist azathioprine, for example. Thus, a hydroxy group is positioned at the chelator-azathioprine conjugate in the finished product. In some embodiments, the tetraazacyclic chelators may be a cyclam or a cyclen, for example. However, the invention is not limited thereto. It should be noted that the hydroxyl group is located at the aliphatic chain of the purine ligands.

In some embodiments, a method of admixing may be carried out in an organic solvent, such as dimethylformamide, acetonitrile, tetrahydrofuran, di-isopropyl ethylamine or a mixture thereof. In some embodiments, one, two, or three of the nitrogen groups of the chelator may be protected, for example, by a tert-butyl or benzyl group, or unprotected.

In some embodiments, the method of the present invention may further include at least one purification step. Any compound of the present invention may be purified via any method known to those of skill in the art. Persons of skill in the art are familiar with such methods, and when those methods may be employed. For example, in a multi-step synthesis that is aimed at arriving at a particular compound, a purification step may be performed after every synthetic step, after every few steps, at various points during the synthesis, and/or at the very end of the synthesis. In some embodiments, one or more purification steps includes technique selected from the group consisting of silica gel column chromatography, HPLC and LC. In certain embodiments, purification methods specifically exclude size exclusion chromatography and/or dialysis. It should be noted that the method of synthesizing the compound in organic solvents and the use of protecting groups, typically offer improvements in the purification of compounds. The installation of protecting groups permits various functional groups of intermediates during the synthesis to be protected, and facilitates the purification of those intermediates. Various means of purification using organic solvents allow for separation and isolation of desired compounds, such as imaging agents, with very little impurities. Thus, it is amenable to develop organic synthetic techniques to allow for site-specific conjugates of higher purities to be obtained in a more efficient way.

In one specific embodiment of the invention, the hydroxypropyl-cyclam or hydroxypropyl-cyclen is conjugated to the azathioprine at one nitrogen group using the synthetic route. In this case, a hydroxy group is incorporated in the finished product. The protected chelator is used as to react a di-bromohydrin to form a chelator-bromohydrin conjugate. The technology platform exploits conjugating antagonists and agonists and seeing their effects in various forms of diseases. In other words, the personalized technology platform may be designed on the basis of individual genetic make-up of enzymatic conversion of purine to DNA proliferation associated to each patient's disease. In other aspects, these synthesis methods may obviate the need of adding protecting groups to purine analogues and increase process efficiency and purify of the final product.

In addition, in some embodiments, pharmaceutical formulations or kits including the compound described above are provided. In other aspects, the compound may be further prepared in the pharmaceutical formulation or the kit using the chemical procedures known to skilled artisans. In some embodiments, the pharmaceutical formulation or the kit may further include antioxidants, stabilizing agents, preservatives or salts, for example. In some embodiments, the pharmaceutical formulation or the kit may include ascorbic acid, mannitol, tin (II) chloride and chelator-azathioprine conjugate, for example. In some aspects, the pharmaceutical formulation or the kit may be an aqueous solution or a solution that has been frozen and/or lyophilized, for example. Herein, the “kit” is also called a “cold kit” or a “drug substance” in the field of molecular imaging.

Furthermore, the present invention accurately provides a method of imaging at the site of a disease in a given subject to perform a per/post treatment evaluation and to be able to monitor that subject for as long as that subject is being treated or under treatment with anti-estrogen. In certain aspects, the method includes detecting a signal generated by the radionuclide-labeled chelator-conjugates at the site of the disease of individual subjects, wherein a site of disease, if present, generates a signal that is more intense than surrounding the tissue. In some aspects, the metal ion may be a radionuclide and any radionuclide known to those of skill in art. In some embodiments, the radionuclides include ^{99m}Tc, ^{67,68}Ga, ^{60,61,62,64,67}Cu, ¹¹¹In, ¹⁶⁶Ho, ^{186,188}Re, ⁹⁰Y, ¹⁷⁷Lu, ²²³Ra, ²²⁵Ac, and ⁸⁹Zr, ^{117m}Sn, ¹⁵³Sm, ⁸⁹Sr, ⁵⁹Fe, ²¹²Bi, ²¹¹At, and ⁴⁵Ti, for example, but the invention is not limited thereto. In other aspects, the metal ion may be a non-radioactive metal. In some embodiments, the site to be imaged may be a tumor or an proteasome-enriched tissue such as XO, HPRT and TPMT over-expressed tissue. In some embodiments, the method may be defined as an imaging method for cancer, rheumatoid arthritis, or bone marrow disease including administration of the compound described above. In one specific embodiment, the method may be defined as a method of imaging a site within a subject including detecting a signal from metal ion labeled chelator-purine ligand conjugate that is localized at the site, but the invention is not limited thereto.

In some embodiments, the signal may be detected using a technique selected from the group consisting of PET, PET/CT, SPECT, SPECT/CT, PET/MRI, SPECT/MRI, and an optical imaging hybrid with nuclear imaging device, for example. In other embodiments, the image may be a gamma image, a PET image, a PET/CT image, a SPECT image, a SPECT/CT image, a PET/MRI image, a SPECT/MRI image, or a hybrid image, for example. It should be noted that the compound described above may be made as a kit for imaging, and an imaging dose is defined as the kit. Besides, the method may be further defined as a method of treating a subject with cancer or chromatin unstable diseases. In particular aspects, the cancer is breast cancer, lung cancer, prostate cancer, ovarian cancer, uterine cancer, cervical cancer, lymphoma or endometrial cancer, for example, but the invention is not limited thereto. In some embodiments, the method may be defined as a treatment method for cancer, rheumatoid arthritis, bone marrow disease, atherosclerosis, or endometrial tissue including administration of the compound described above, for example. In other words, there is provided a method of imaging a site, diagnosing a disease, or treating a disease within a subject including administering a metal ion labeled-chelator-purine ligand conjugate to the subject, wherein the site is imaged, the disease is diagnosed, or the disease is treated.

On the other hand, the compound of the invention may be applied to molecular imaging and therapy. For example, the compound of the invention may be used as a molecular nuclear imaging agent. Specifically, the molecular nuclear imaging agent enables the comprehensive characterization of therapeutic intervention and can be used in patient selection, pharmacokinetic, dosage-finding and proof-of-concept studies. The effort in purine image-guided cell therapy approaches in parallel with instrumentation development would be more comprehensive in the outcome assessment of patient response to treatment. More specifically, the molecular imaging agent using chelation provides advantages in batch-to-batch reproducibility of radiochemical yield, purity, production cost and the availability of the agent in routine clinical practice.

In addition, the invention technology platform integrates a metal ion, chelator, and purine ligand. The purine ligand may be used as a homing agent, which plays a dual role by cross talking between cell surface transporters and intracellular proteasomes, thus, enhance cell uptake of the homing agent. Such a purine-based imaging would help to monitor purine pathway-directed treatment response as well as predict the selection of patients for optimal treatment response. In this case, a hydroxy group was incorporated at the aliphatic spacers in chelator-azathioprine conjugates to allow phosphorylation during diagnostic imaging with innovative tools to understand the dynamic changes in pathway-activated enzymatic systems leading to tissue degeneration, inflammatory, and proliferative disorders and to improve patient diagnosis, therapy and prognosis. However, the invention is not limited thereto.

To prove that the compounds of the present invention are suitable for imaging and be used for cancer therapy, the compounds of the present invention are synthesized and tested by using the method described in the following examples.

### Example 1. Synthesis of Compound SC-06-L-1

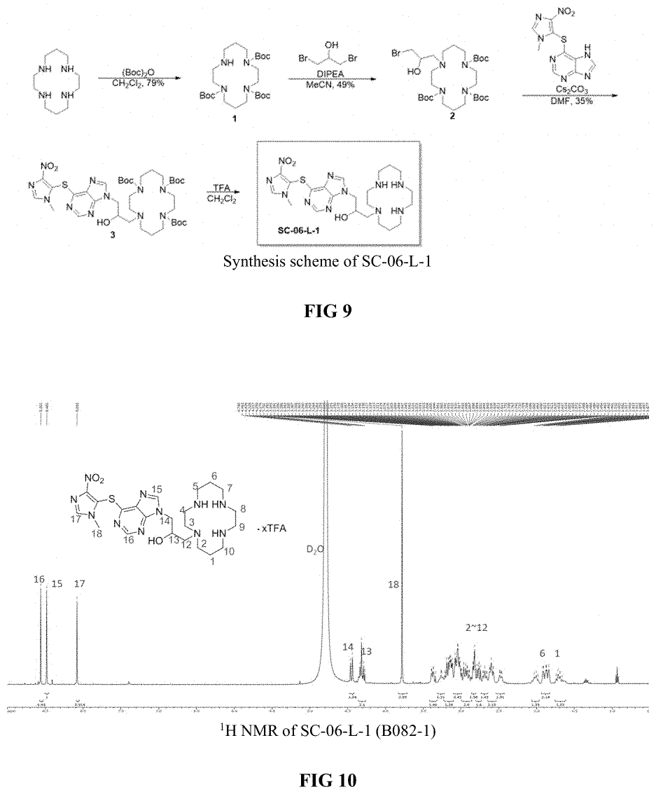

In this example, SC-06-L-1 of the present invention was synthesized in 3 specific steps. The synthetic scheme is shown in FIG. 9. All compounds were analyzed by NMR, mass spectrometer, and HPLC. NMR data was collected from 500 MHz Varian Inova NMR spectrometer (Palo Alto, Calif.) equipped with 5 mm PFG Triple ¹H-¹³C-¹⁵N probe, 5 mm PFG ¹H-¹⁹C-¹⁵N-³¹P switchable probe and 4 mm ¹H-¹³C Nano probe. Mass Spectrometry was obtained from Bruker Solarix (Germany). HPLC data was collected from Waters 2695 Separations Module (Milford, Mass.) equipped with PC HILIC Column, (5 μm, 2.0 mm I.D.×150 mm).

Step 1. Synthesis of Protected Cyclam-Bromohydrin Conjugate (Compound 2)

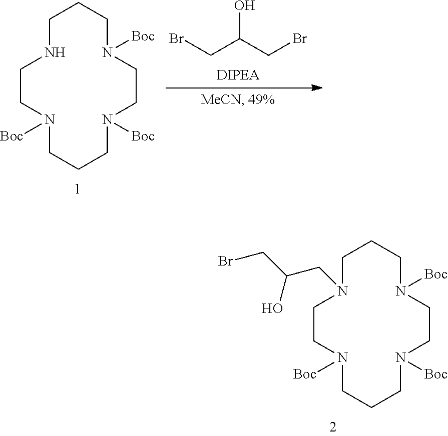

Diisopropylethylamine (DIPEA, 7.34 mL, 42.16 mmol) was added dropwise to a solution of tri-BOC protected compound 1 (1.41 g, 2.81 mmol) and acetonitrile (MeCN, 20 mL) at room temperature (r.t.). The mixture was stirred at r.t. for 30 min and 1,3-dibromo-2-propanol (2.87 mL, 28.11 mmol) was then added dropwise. The reaction was warmed and stirred at reflux for 18 h. The reaction was cooled to r.t. and the solvent was removed under reduced pressure. The crude product was purified by column chromatography (ethyl acetate:hexane, 1/1) to give compound 2 (869.8 mg, 1.36 mmol, 49%) as a white solid.

Step 2. Synthesis of Protected Cyclam-Hydroxypropyl-Azathioprine Conjugate (Compound 3)

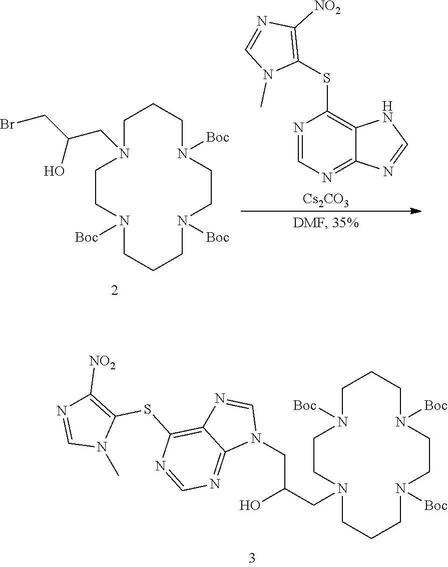

Cesium carbonate (Cs₂CO₃, 54.5 mg, 0.17 mmol) was added to a solution of azathioprine (23.2 mg, 0.08 mmol) and dimethylformamide (DMF, 5 mL) at r.t. The mixture was stirred at r.t. for 30 min and a solution of compound 2 (106.7 mg, 0.17 mmol) in DMF (3 mL) was added dropwise. The reaction was warmed and stirred at 100° C. for 18 h. The reaction was cooled to r.t., EA (150 mL) was added to reaction and the reaction was extracted with water three times (150 mL×3). The organic layer was dried over magnesium sulfate, filtered and concentrated. The crude product was purified by column chromatography (5% ethyl acetate in methanol) to give compound 3 (24.3 mg, 0.03 mmol, 35%) as a yellow solid.

Step 3. Synthesis of Cyclam-Hydroxypropyl-Azathioprine Conjugate (Compound SC-06-L-1)

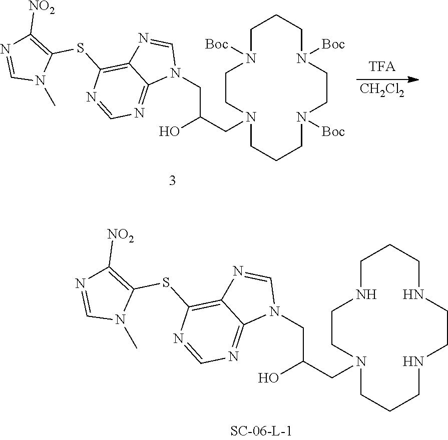

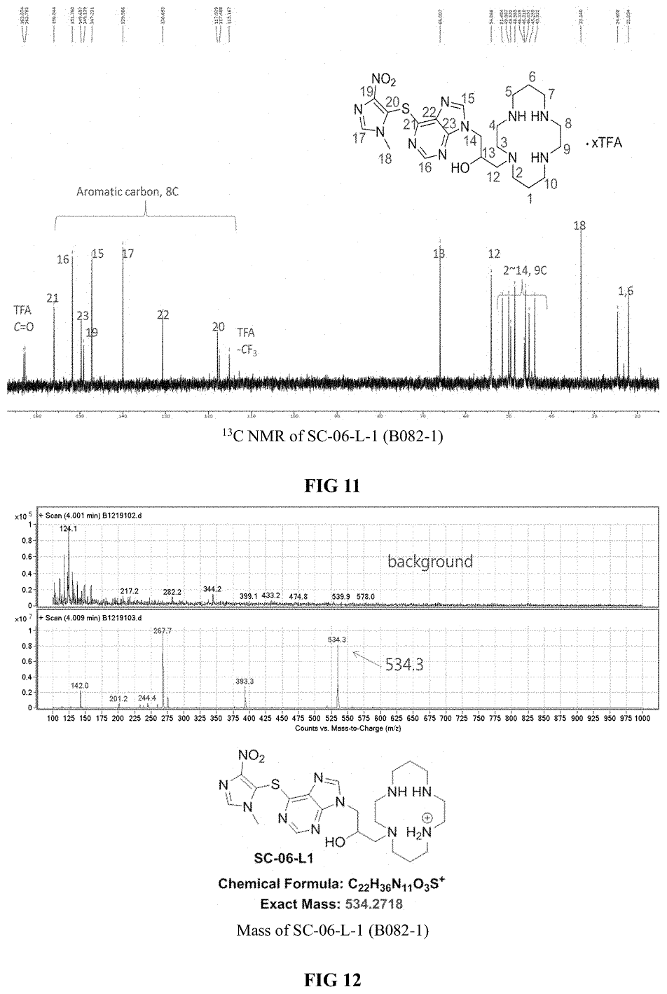

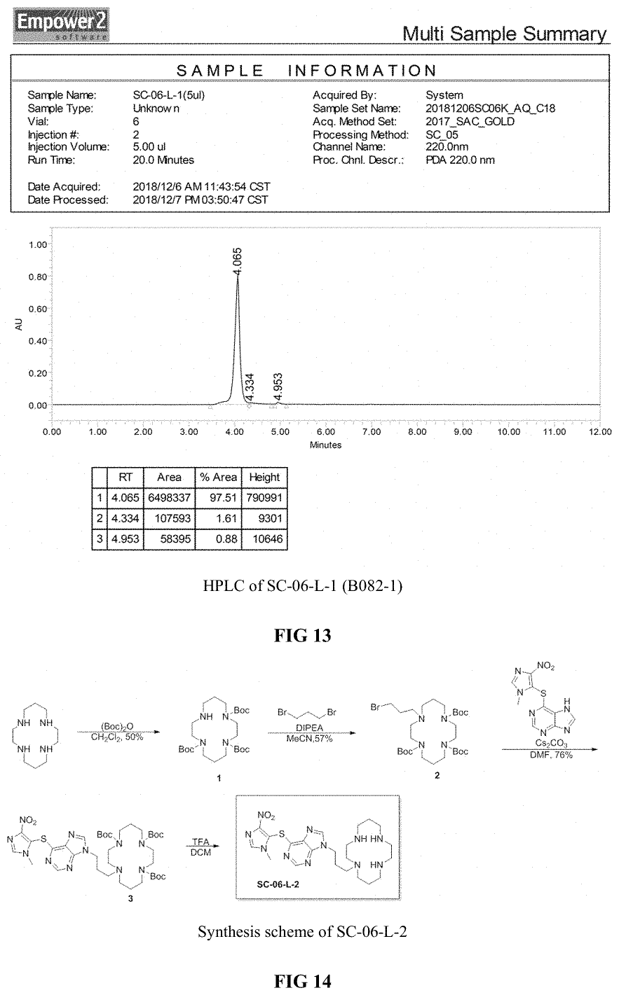

Trifluoroacetic acid (TEA, 3 mL) was added dropwise to a solution of compound 3 (96 mg, 0.12 mmol) dissolved in dichloromethane (CH₂Cl₂, 6 mL) in ice-bath. The mixture was stirred at r.t. for 18 h. The solvent and reagent were removed under reduced pressure. The crude product was purified by reverse phase column chromatography to give SC-06-L-1 (80.5 mg) as a white solid. The structure of Compound SC-06-L-1 was confirmed by ¹H-NMR and ¹³C-NMR, and the analysis results are presented in FIG. 10 to FIG. 11, respectively. Also, Compound SC-06-L-1 was analyzed using mass spectrometry and HPLC and the results are presented in FIG. 12 and FIG. 13.

### Example 2. Synthesis of Compound SC-06-L-2

In this example, SC-06-L-2 of the present invention was synthesized in 3 specific steps. The synthetic scheme is shown in FIG. 14.

Step 1. Synthesis of Protected Cyclam-Bromopropyl Conjugate (Compound 2)

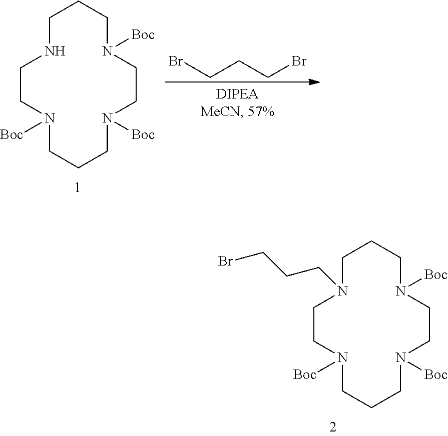

Diisopropylethylamine (2 mL, 11.48 mmol) was added dropwise to a solution of compound 1 (385.3 mg, 0.77 mmol) and acetonitrile (8 mL) at r.t. The mixture was stirred at r.t. for 30 min and 1,3-dibromopropane (0.78 mL, 7.69 mmol) was added dropwise. The reaction was warmed and stirred at reflux for 18 h. The reaction was cooled to r.t. and the solvent was removed under reduced pressure. The crude product was purified by column chromatography (EA/hexane, 1/2) to give compound 2 (270.5 mg, 0.435 mmol, 57%) as a white solid.

Step 2. Synthesis of Protected Cyclam-Propyl-Azathioprine Conjugate

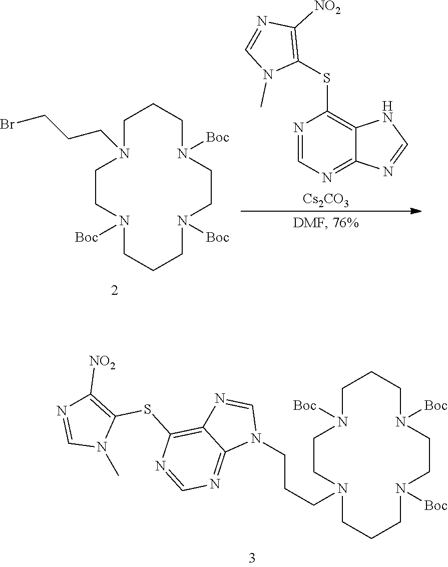

Cesium carbonate (178.2 mg, 0.55 mmol) was added to a solution of azathioprine (79.5 mg, 0.29 mmol) and DMF (2 mL) at r.t. The mixture was stirred at r.t. for 30 min and a solution of compound 2 (340 mg, 0.55 mmol) in DMF (4 mL) was added dropwise. The reaction was warmed and stirred at 100° C. for 18 h. The reaction was cooled to r.t., EA (150 mL) was added to reaction and the reaction was extracted with water three times (150 mL×3). The organic layer was dried over magnesium sulfate, filtered and concentrated. The crude product was purified by column chromatography (5% EA in Methanol) to give compound 3 (177.4 mg, 0.22 mmol, 76%) as a yellow solid.

Step 3. Synthesis of Cyclam-Propyl-Azathioprine Conjugate (Compound SC-06-L-2)

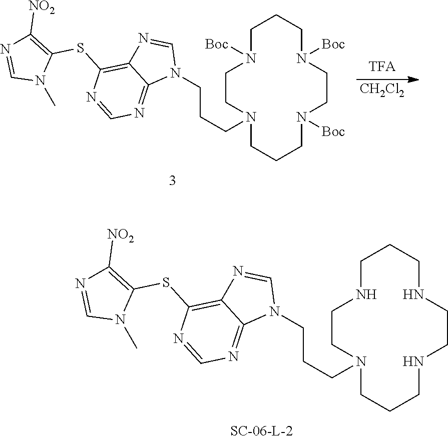

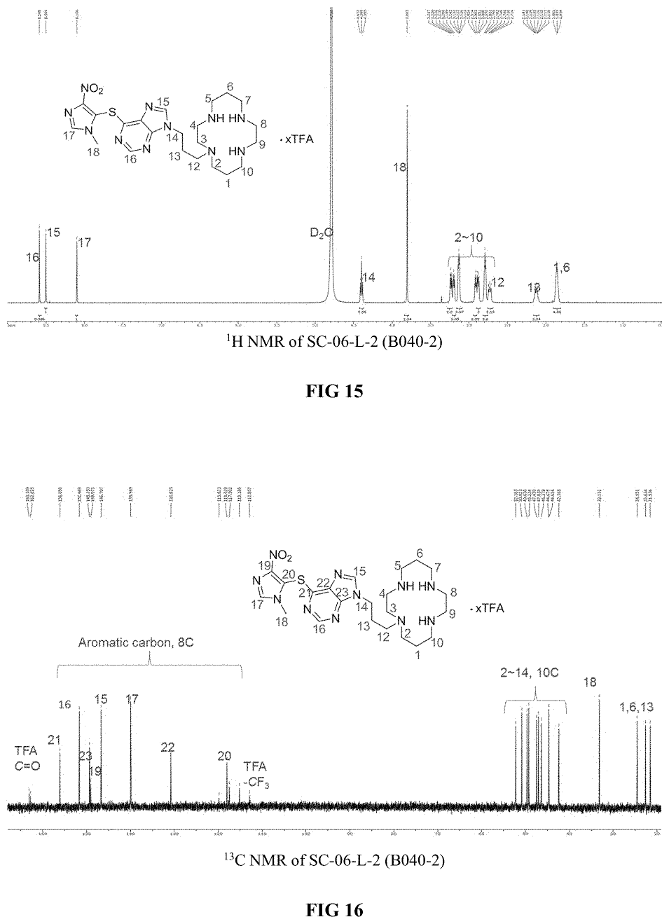

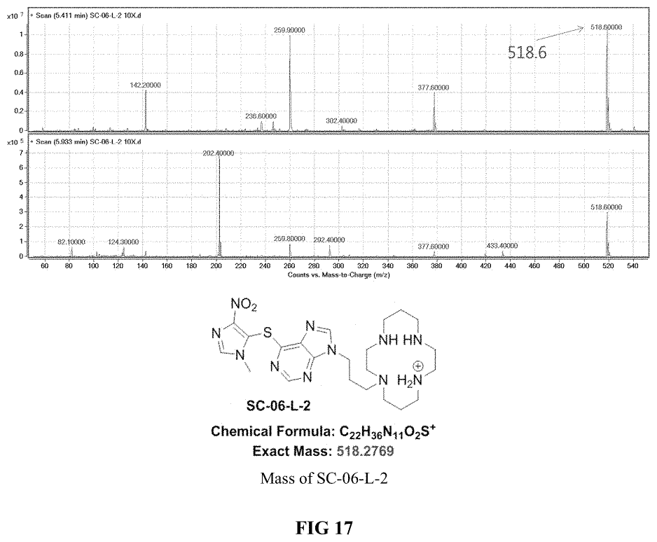

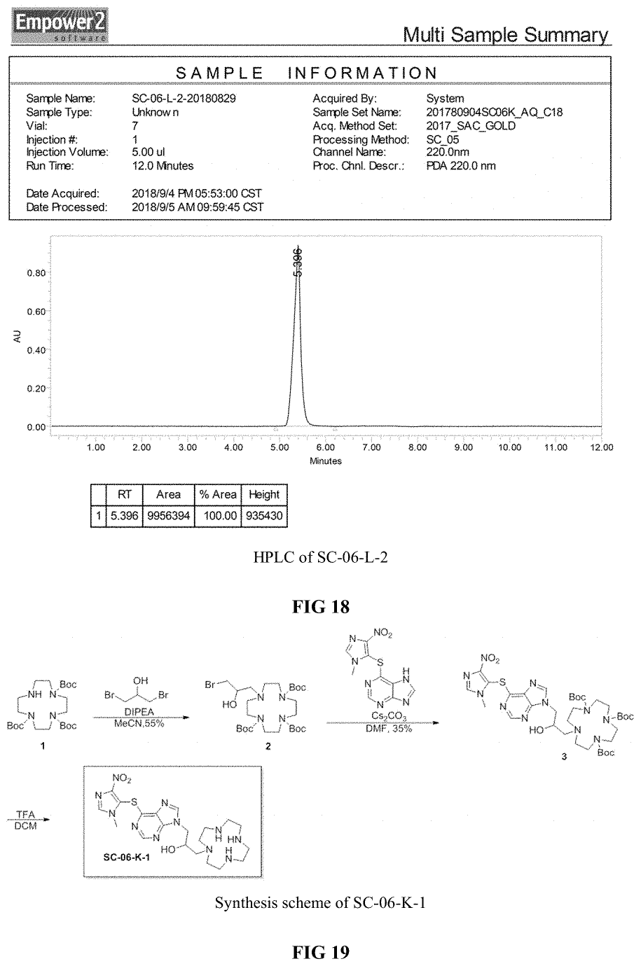

Trifluoroacetic acid (3 mL) was added dropwise to a solution of compound 3 (177.4 mg, 0.22 mmol) and dichloromethane (6 mL) in ice-bath. The mixture was stirred at r.t. for 18 h. The solvent and reagent were removed under reduced pressure. The crude product was purified by reverse phase column chromatography to give SC-06-L-2 (80.8 mg) as a white solid. The structure of Compound SC-06-L-2 was confirmed by ¹H-NMR and ¹³C-NMR, and the analysis results are presented in FIG. 15 to FIG. 16, respectively. Also, Compound SC-06-L-2 was analyzed using mass spectrometry and HPLC and the results are presented in FIG. 17 and FIG. 18.

### Example 3. Synthesis of Compound SC-06-K-1

In this example, SC-06-K-1 of the present invention was synthesized in 3 specific steps. The synthetic scheme is shown in FIG. 19.

Step 1. Synthesis of Protected Cyclen-Bromohydrin Conjugate (Compound 2)

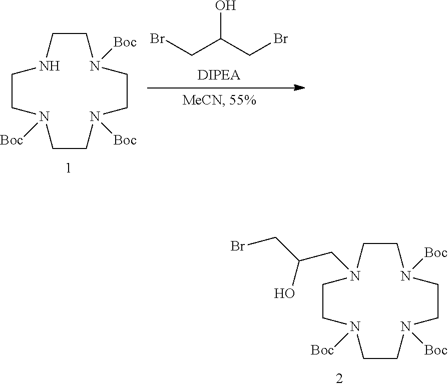

Diisopropylethylamine (6.79 mL, 39 mmol) was added to a solution of compound 1 (1.23 g, 2.6 mmol) and acetonitrile (20 mL) at r.t. dropwise. The mixture was stirred at r.t. for 30 min and 1,3-dibromo-2-propanol (2.65 mL, 26 mmol) was added dropwise. The reaction was warmed and stirred at reflux for 18 h. The reaction was cooled to r.t. and the solvent was removed under reduced pressure. The crude product was purified by column chromatography (EA/hexane, 1/1) to give compound 2 (863.1 mg, 1.42 mmol, 55%) as a white solid.

Step 2. Synthesis of Protected Cyclen-Hydroxypropyl-Azathioprine Conjugate (Compound 3)

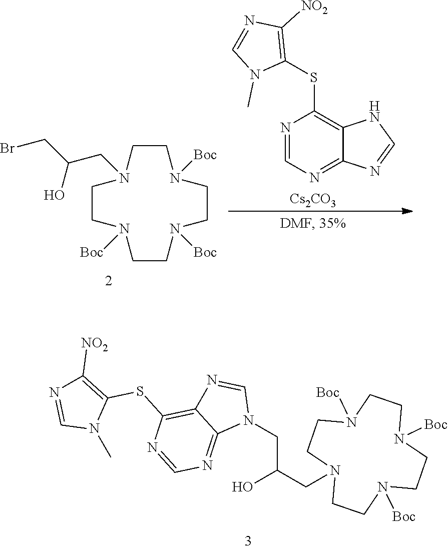

Cesium carbonate (877.7 mg, 2.69 mmol) was added to a solution of azathioprine (373.5 mg, 1.35 mmol) and DMF (5 mL) at r.t. The mixture was stirred at r.t. for 30 min and a solution of 2 (1.64 g, 2.69 mmol) in DMF (4 mL) was added dropwise. The reaction was warmed and stirred at 100° C. for 18 h. The reaction was cooled to r.t., EA (150 mL) was added to reaction and the reaction was extracted with water three times (150 mL×3). The organic layer was dried over magnesium sulfate, filtered and concentrated. The crude product was purified by column chromatography (5% EA in Methanol) to give compound 3 (278.5 mg, 0.34 mmol, 35%) as a yellow solid.

Step 3. Synthesis of Cyclen-Hydroxypropyl-Azathioprine Conjugate (Compound SC-06-K-1)

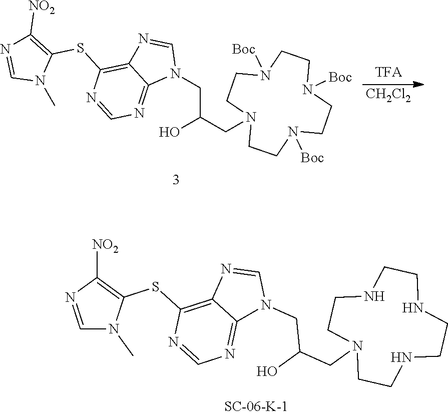

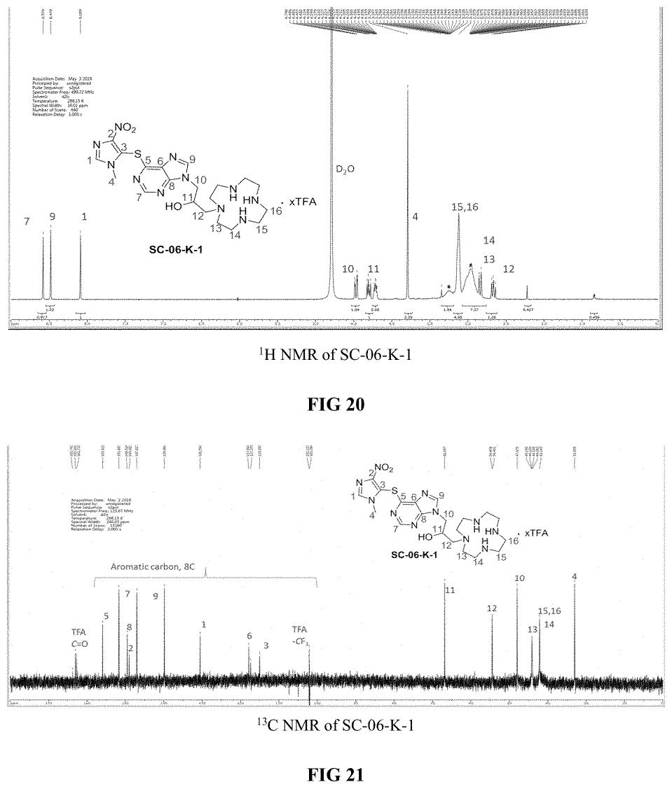

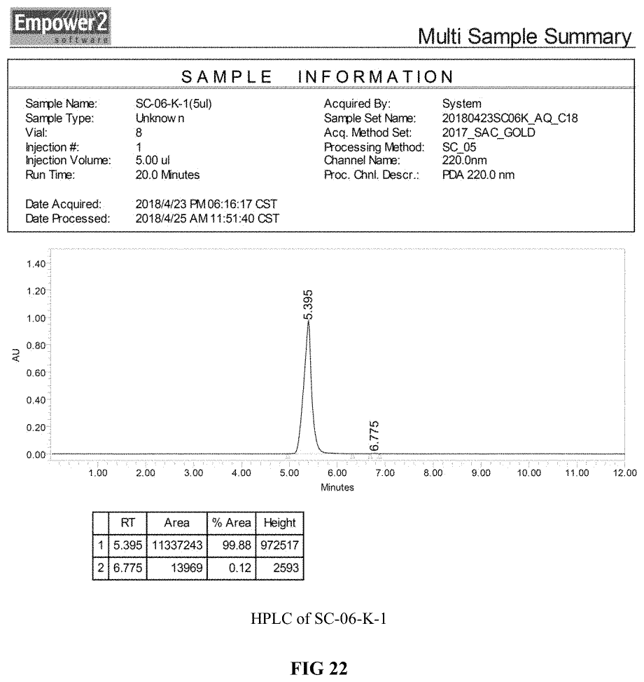

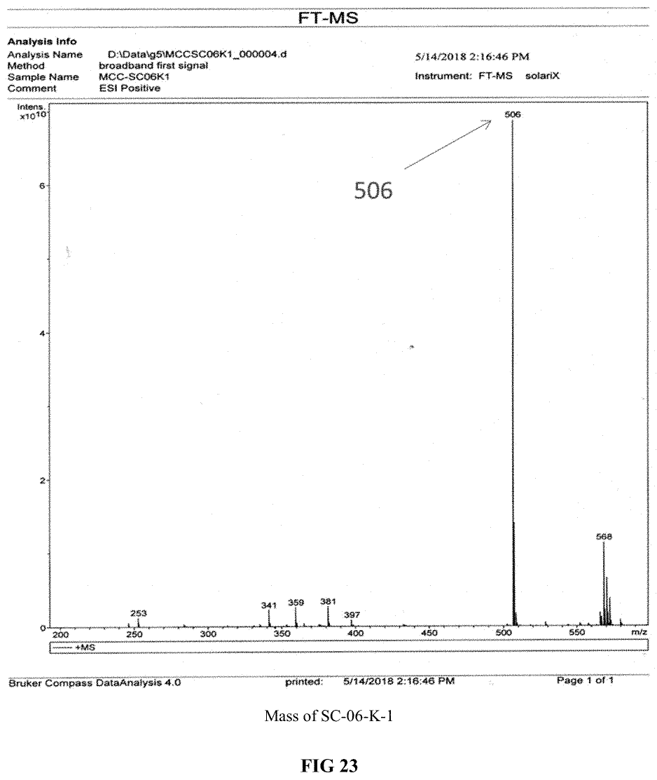

Trifluoroacetic acid (3 mL) was added dropwise to a solution of compound 3 (151.8 mg, 0.19 mmol) and dichloromethane (6 mL) in an ice-bath. The mixture was stirred at r.t. for 18 h. The solvent and reagent were removed under reduced pressure. The crude product was purified by reverse phase column chromatography to give SC-06-K-1 (108.8 mg) as a white solid. The structure of Compound SC-06-K-1 was confirmed by ¹H-NMR and ¹³C-NMR, and the analysis results are presented in FIG. 20 to FIG. 21, respectively. Also, Compound SC-06-L-1 was analyzed using mass spectrometry and HPLC and the results are presented in FIG. 22 and FIG. 23.

### Example 4. Synthesis of Compound SC-06-K-2

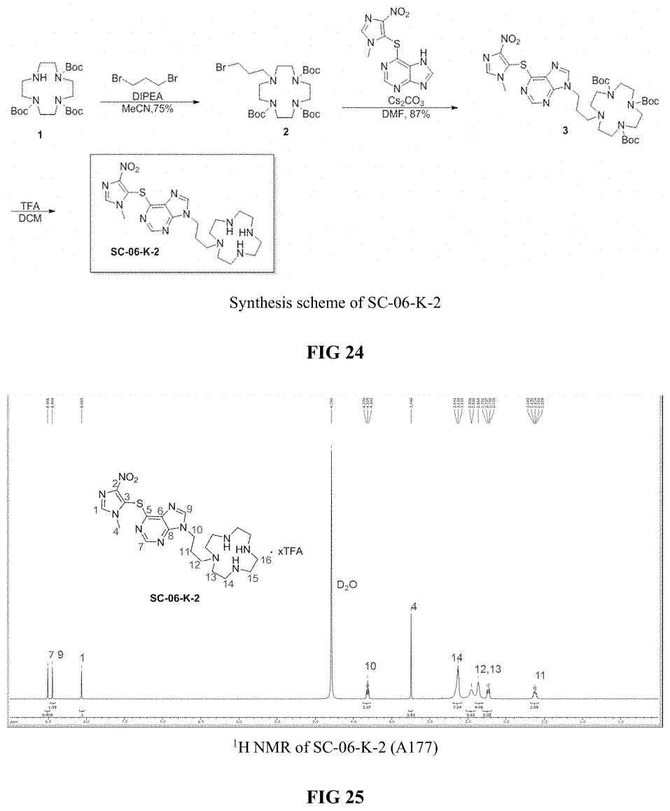

In this example, SC-06-K-2 of the present invention was synthesized in 3 specific steps. The synthetic scheme is shown in FIG. 24.

Step 1. Synthesis of Protected Cyclen-Bromopropyl Conjugate (Compound 2)

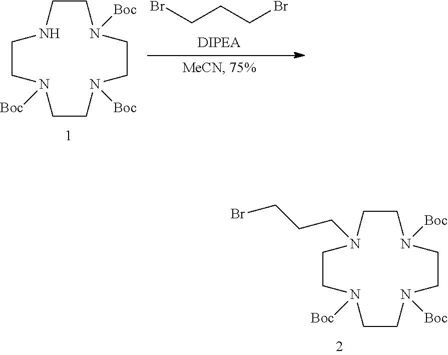

Diisopropylethylamine (9.23 mL, 53 mmol) was added to a solution of compound 1 (1067 g, 3.53 mmol) and acetonitrile (15 mL) at r.t. dropwise. The mixture was stirred at r.t. for 30 min and 1,3-dibromopropane (3.5 mL, 35.3 mmol) was added dropwise. The reaction was warmed and stirred at reflux for 18 h. The reaction was cooled to r.t. and the solvent was removed under reduced pressure. The crude product was purified by column chromatography (EA/hexane, 1/2) to give compound 2 (1.6 g, 2.65 mmol, 75%) as a white solid.

Step 2. Synthesis of Protected Cyclen-Propyl-Azathioprine Conjugate

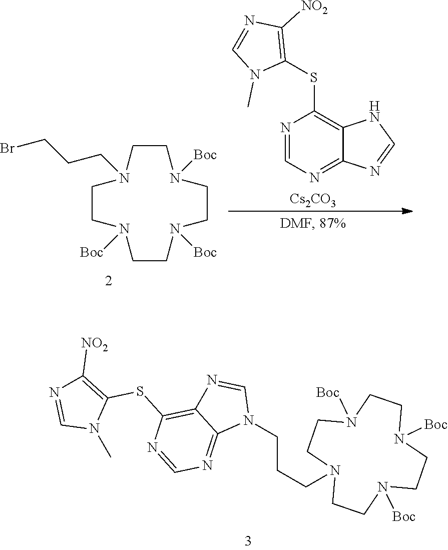

Cesium carbonate (863 mg, 2.65 mmol) was added to a solution of azathioprine (367.4 mg, 1.33 mmol) and DMF (5 mL) at r.t. The mixture was stirred at r.t. for 30 min and a solution of compound 2 (1.6 g, 2.65 mmol) in DMF (4 mL) was added dropwise. The reaction was warmed and stirred at 100° C. for 18 h. The reaction was cooled to r.t., EA (150 mL) was added to reaction and the reaction was extracted with water three times (150 mL×3). The organic layer was dried over magnesium sulfate, filtered and concentrated. The crude product was purified by column chromatography (5% EA in Methanol) to give compound 3 (908.5 mg, 1.15 mmol, 87%) as a yellow solid.

Step 3. Synthesis of Cyclen-Propyl-Azathioprine Conjugate (Compound SC-06-K-2)

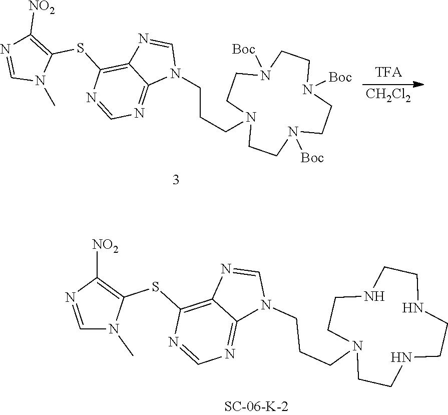

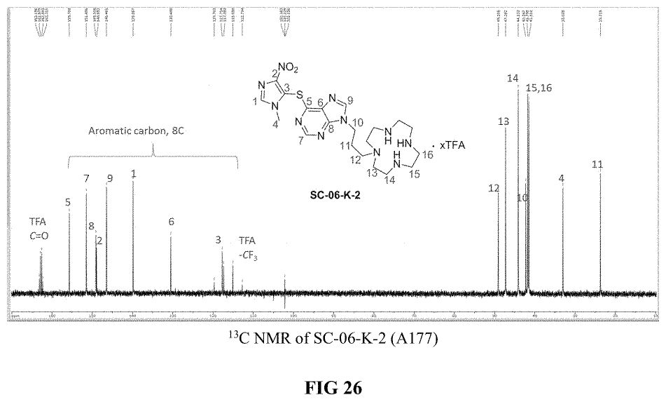

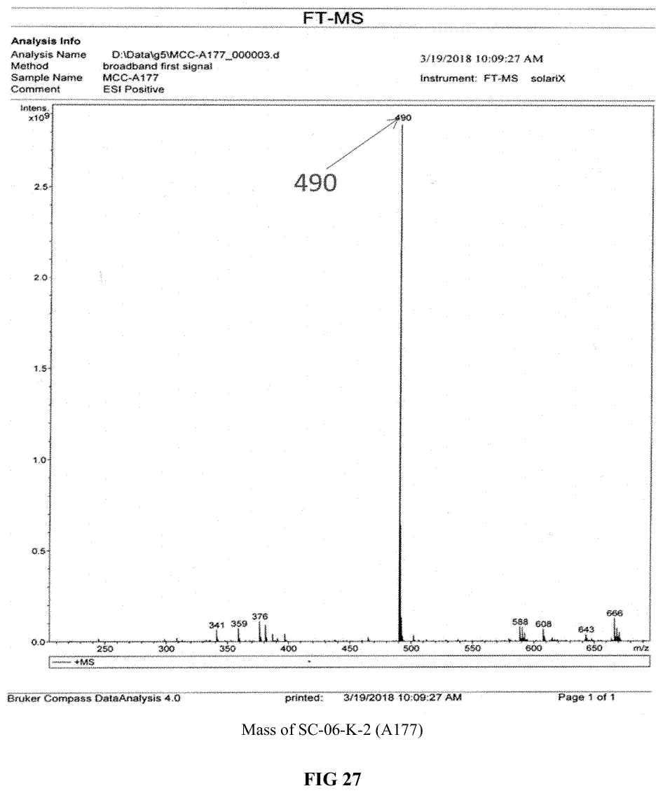

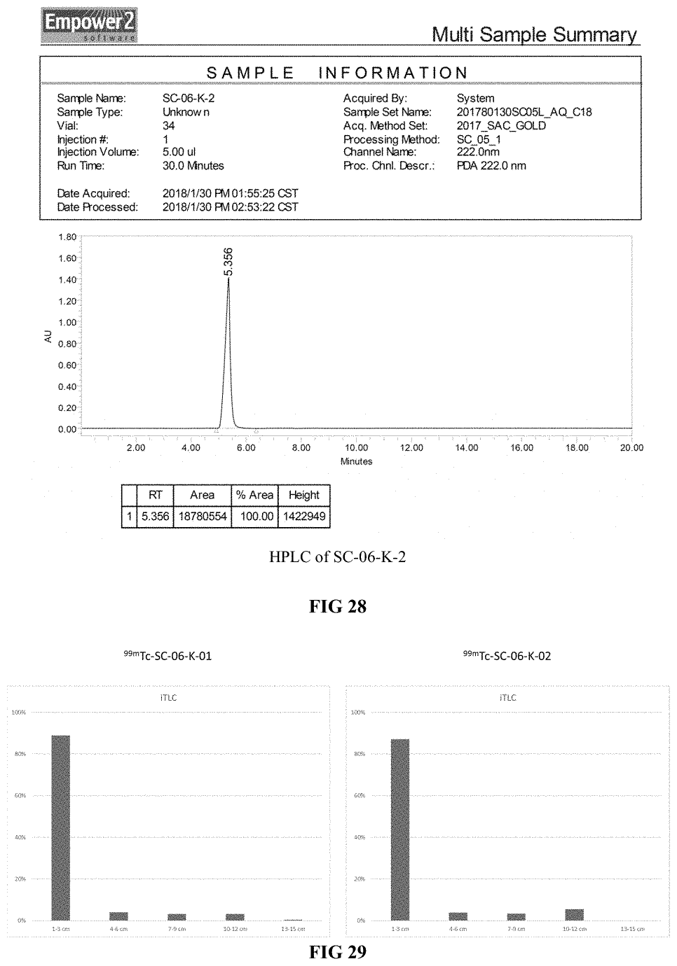

Trifluoroacetic acid (3 mL) was added dropwise to a solution of compound 3 (454.3 mg, 0.58 mmol) and dichloromethane (6 mL) in an ice-bath. The mixture was stirred at r.t. for 18 h. The solvent and reagent were removed under reduced pressure. The crude product was purified by reverse phase column chromatography to give SC-06-K-2 (247.5 mg) as a white solid. The structure of Compound SC-06-K-2 was confirmed by ¹H-NMR and ¹³C-NMR, and the analysis results are presented in FIG. 25 to FIG. 26, respectively. Also, Compound SC-06-L-2 was analyzed using mass spectrometry and HPLC and the results are presented in FIG. 27 and FIG. 28.

### Example 5. General Manufacturing of Compound ^{99m}Tc-SC-06 Analogues

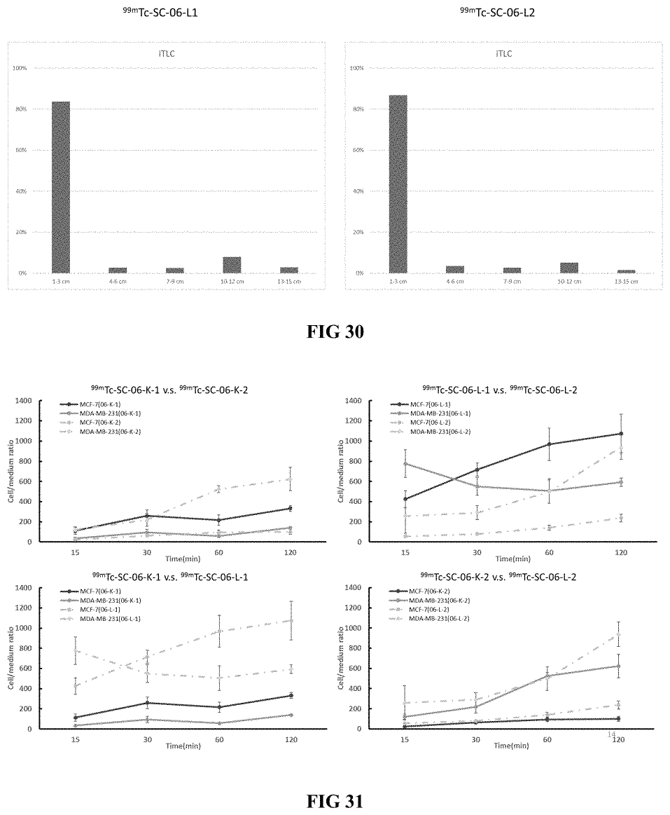

Sodium pertechnetate (Na^{99m}TcO₄) was obtained from ⁹⁹Mo/^{99m}Tc generator by FRI (Japan). Radiosynthesis of Compound ^{99m}Tc-SC-06 analogue was achieved by adding ^{99m}Tc-pertechnetate (1 mCi, 0.14 mL) into the lyophilized residue of Compound SC-06-L-1 (5 mg in 1 mL water) and tin (II) chloride (SnCl₂, 100 μg in 0.1 mL water). The complexation of Compound SC-06 analogue with ^{99m}Tc was carried out at pH 6.5 for 5 min. The product was reconstituted in 1 mL and filtered through a 0.22 um filter to assure sterility. Radiochemical purity was determined by TLC (Waterman No. 1, Aldrich-Sigma, St. Louis, Mo.) eluted with saline. FIG. 29 to FIG. 30 showed the radiochemical purities of Compound ^{99m}Tc-SC-06-K-1 and K-2, ^{99m}Tc-SC-06-L-1 and L-2 synthesized in Example 2 of the invention. The radiochemical purities of these four compounds were greater than 85%, with Rf value 0.1 using saline as a mobile phase. For mouse imaging studies, 0.1 mL (100 ug SC-06-L1 in 0.1 mCi of ^{99m}Tc) was injected intravenously via tail vein into mice.

### Example 6. In Vitro Cellular Uptake Studies

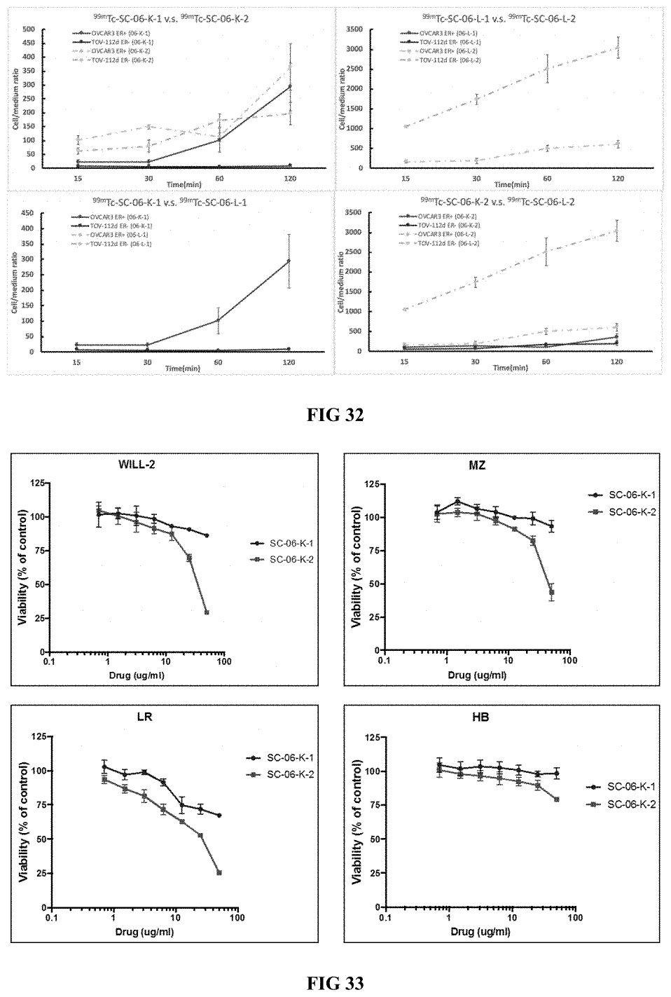

A 6-well plate was used for breast cancer cells (MDA-MB-231, MCF-7), OVCAR3 (epithelial ovarian cancer cells) and TOV-112D (ascites ovarian cancer cells) cell uptake studies. Each well contained 100,000 cells in 150 μL serum free RPMI. Compound ^{99m}Tc-SC-06-K analogues (dose: 0.1 mg/0.1 mCi/20 μL/well) were added to each well containing cells in the culture medium for different intervals (0-2 hr). Subsequently, cells are washed with ice-cold phosphate-buffered saline PBS twice and trypsinized with 0.5 mL of trypsin solution to detach tumor cells. Protein concertation assay was used to determine the proteins in each well. The cells were lysed in the lysis buffer containing proteinase inhibitors (Roche Diagnostic, Mannheim, Germany). The protein concentration in the cell lysate was quantified using Bradford Method as described by the manufacture (Bio-RAD, Hercules, Calif., USA). The Bradford dye was diluted in distilled water (1:4) and filtered through filter paper (number 1, Whatman no. 1, Advantec Co. Ltd., Tokyo). Bovine serum albumin at the concentration of 1000 μg/ml, 500 μg/ml, 250 μg/ml, 125 μg/ml, 62.5 g/ml, 31.25 μg/ml were used to build a standard curve. Protein samples were diluted in lysis buffer at 1:9. Diluted protein samples or standard were mixed with Bradford dye in 96 well, then the absorbance at 595 nm was recorded. The radioactivity concentration in the cells and culture medium was measured with a gamma counter (Packard, CT) and expressed as cpm/g of cells and cpm/g medium. The protein mass-to-medium radioactivity concentration ratio was calculated and plotted over time. In Vitro cell/media ratios (volume of distribution) in breast cancer cells showed that ^{99m}Tc-SC-06-L1 had high uptake in time-dependent patterns which had potential for imaging breast tumors (FIG. 31). In ovarian cancer cells, ^{99m}Tc-SC-06 analogues had high cell/media ratios in time-dependent patterns which had potential for imaging ovarian tumors (FIG. 32).

### Example 7. In Vitro Anti-Cancer Studies

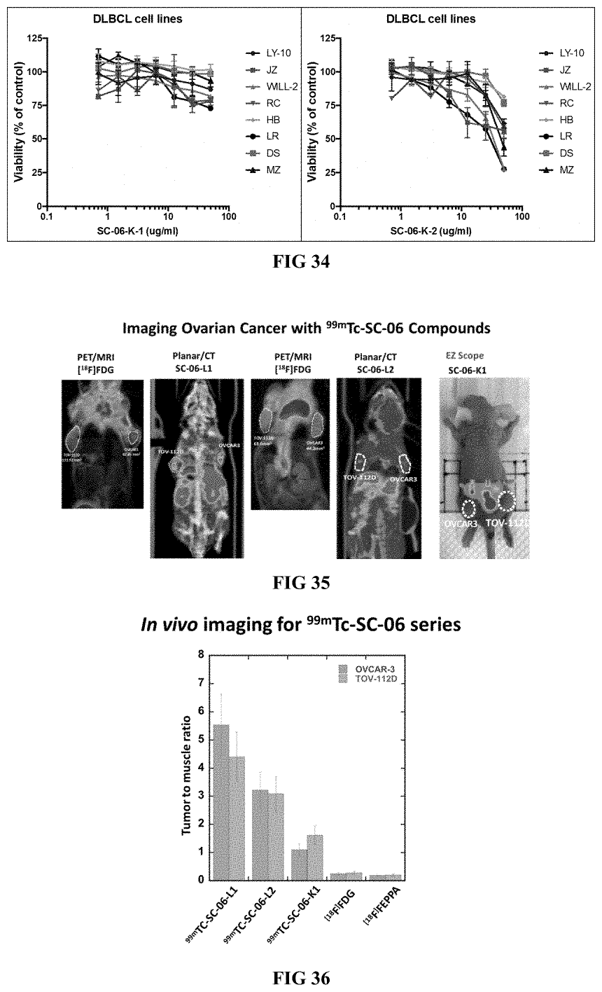

Effect of Compound SC-06-K analogues were selected to test their feasibility against representative mantle cell lines and diffuse large B-cell lymphoma (DLBCL) cell lines using cell viability assays. In Vitro MTT cytotoxicity assays of SC-06-K analogues against various human lymphoma cells indicating SC-06-K-1 inhibited cancer cells in dose-dependent manners (FIG. 33). SC-06-K-1 inhibited various human diffuse large B-cell lymphoma cells in a dose-dependent manner (FIG. 34).

### Example 8. Molecular Imaging of Ovarian Tumors and Breast Tumors

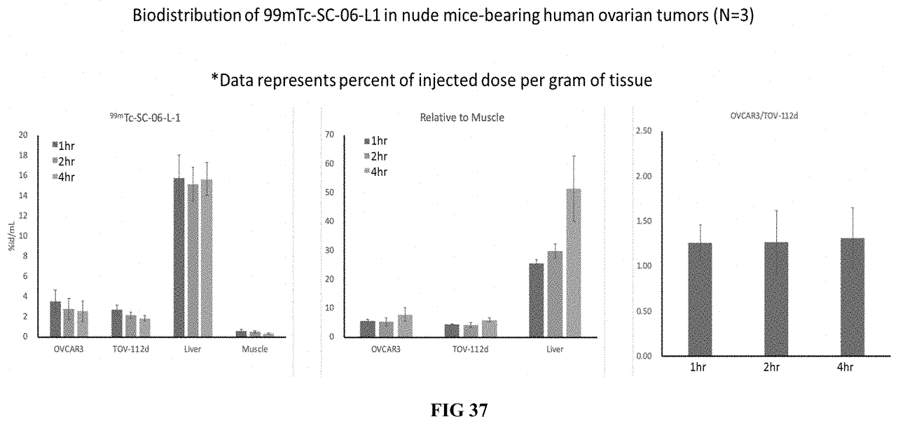

An athymic nude mouse was inoculated with human ovarian tumors (OVCA-3 and TOV-112d) under both armpits in front legs. When tumor reached 0.5 cm, the mouse was administered with either ^{99m}Tc-SC-06-L1 or ¹⁸F-FDG (standard). The images were collected by planar/CT or PET/MRI. CT and MRI were used to attenuate anatomical locations. ^{99m}Tc-SC-06-L1 and ^{99m}Tc-SC-06-L2 Planar/CT revealed higher uptake in an athymic nude mouse-bearing human ovarian tumors (OVCA-3 and TOV-2d) whereas ¹⁸F-FDG had poor uptake (FIG. 35). Compare to PET/MRI ¹⁸F-FDG, SC-06 compounds showed significant increased uptake in tumors. ^{99m}Tc-SC-06-L1 demonstrated the superior pharmacokinetic properties and high contrast visualization in ovarian tumors (FIG. 36). Biodistribution of ^{99m}Tc-SC-06-L1 in nude mice-bearing human ovarian tumors showed OCAR-3 tumor had more uptake than TOV-112d (FIG. 37).

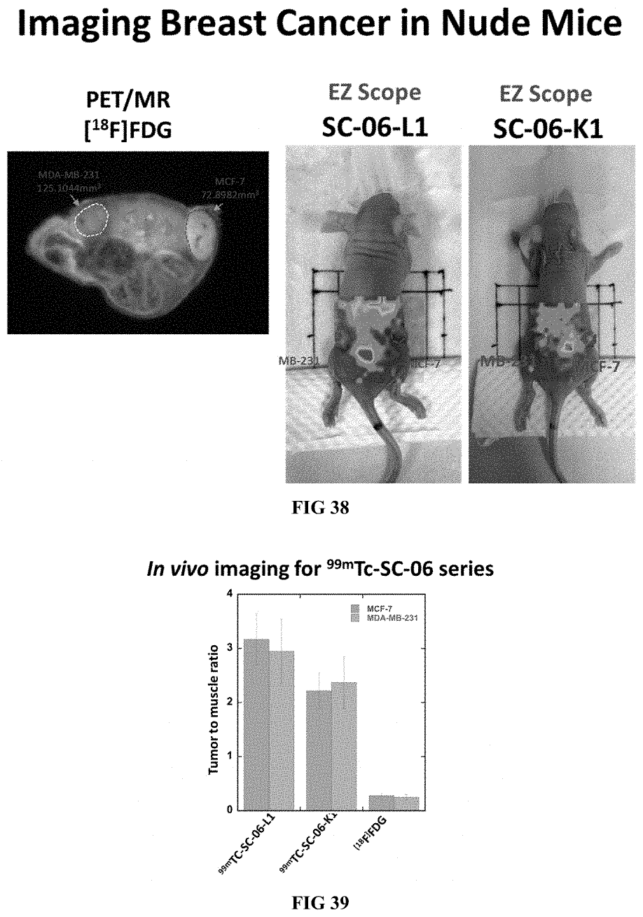

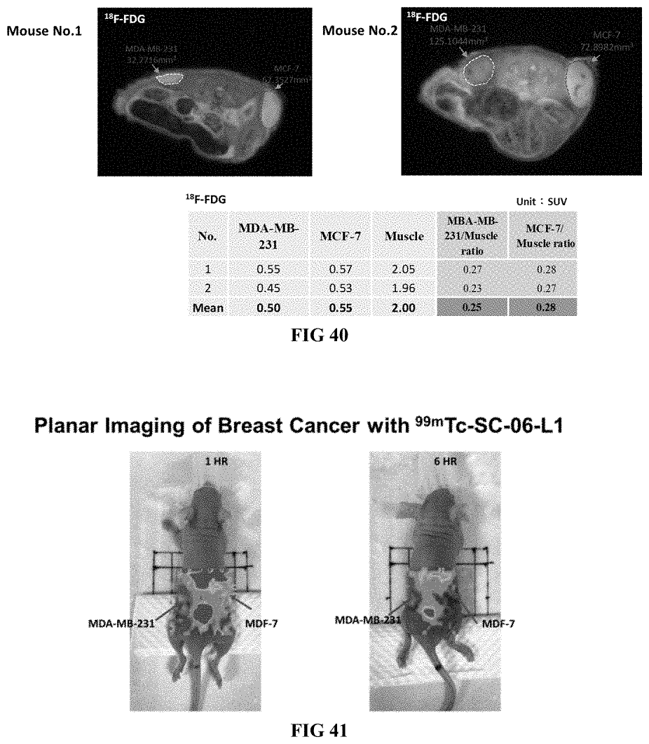

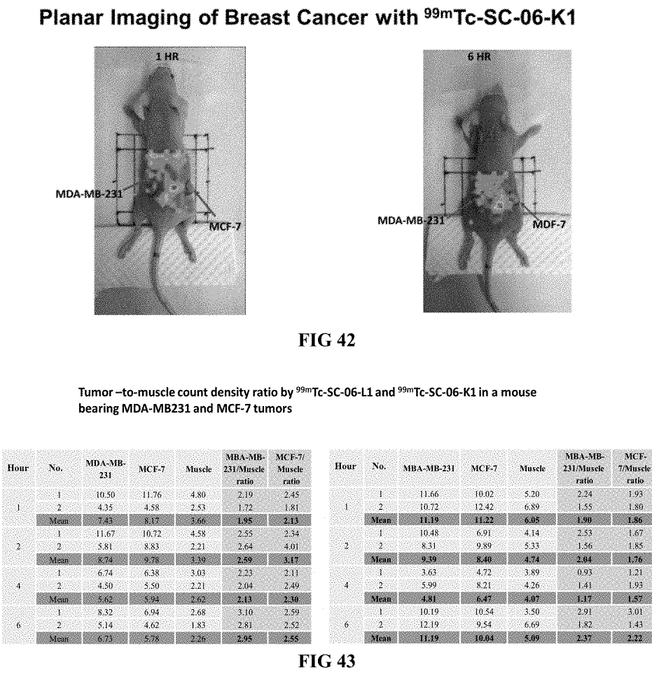

Athymic nude mouse was inoculated with human ovarian tumors (MDA-MB231 and MCF-7). When tumor reached 0.5 cm, the mice were administered with either ^{99m}Tc-SC-06-L1, ^{99m}Tc-SC-06-K1 and ¹⁸F-FDG (standard) (120 uCi/100 uL per mouse, n=2/compound, iv). The images were acquired by PET/MRI at 30 min and eZ-Scope (Anzai Medical Compant, Japan) at 1-6 hrs. ^{99m}Tc-SC-06-L1 and ^{99m}Tc-SC-06-K1 showed high uptake in nude mice bearing MDA-MB231 and MCF tumors compared to ¹⁸F-FDG (FIG. 38). Compare to ¹⁸F-FDG, SC-06 compounds showed significant increased uptake in tumors. ^{99m}Tc-SC-06-L1 demonstrated the superior pharmacokinetic properties and high contrast visualization in breast tumors (FIG. 39). In nude mice administered with ¹⁸F-FDG, tumor/muscle count density ratios at 0.5 hrs were 0.25 (MDA-MB-231) and 0.28 (MCF-7), respectively (FIG. 40). However, tumor/muscle count density ratios by ^{99m}Tc-SC-06-L1 and ^{99m}Tc-SC-06-k1 in both MDA-MB231 and MCF tumors at 1-6 hrs ranged 2.0-3.2 and 1.2-2.4, respectively (FIG. 39 to FIG. 43). Tumor-to-background count density ratios for ^{99m}Tc-SC-06-K1 and ^{99m}Tc-SC-06-L1 were higher than ¹⁸F-FDG in both tumor types indicating that ^{99m}Tc-SC-06-L1 and ^{99m}Tc-SC-06-k1 had better sensitivity than ¹⁸F-FDG in tumor detection (FIG. 40 to FIG. 42). In MCF-7 and MB-231 tumor-bearing animal model, ^{99m}Tc-SC-06-L1 showed slightly tumor/muscle count density ratios than ^{99m}Tc-SC-06-k1 (FIG. 43). The findings indicate ^{99m}Tc-SC-06-K1 and ^{99m}Tc-SC-06-L1 are sensitive markers for DNA proliferation that have potential to image various types of tumors.

In summary, the present invention provides the compound to quantify enzymatic conversion activities in purine pathway-directed systems. The hydroxy group is incorporated in the finished product. In the compound of the present invention, the protected bromohydrin chelator is used as to react the thiopurine ligand to form the chelator-purine ligand conjugate. The technology platform may exploit conjugating antagonists and agonists (biomarkers) and seeing their effects in various forms of diseases. Also, the compound may be further prepared in pharmaceutical formulations and kits using the chemical procedures known to skilled artisans. In addition, the method of synthesizing the compound is also provided. Besides, the compound of the present invention may be used for imaging or treating purine pathway associated diseases.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

### REFERENCE CITED

1. Kubota K, Ishiwata K, Kubota R, Yamada S, Tada M, Sato T, Ido T. Tracer feasibility for monitoring tumor radiotherapy: A quadruple tracer study with fluorine-18-fluorodeoxyglucose or fluorine-18-fluorodeoxyuridine, L-[Methyl-¹⁴C]methionine,[6-³H]thymidine, and gallium-67. J Nucl Med. 1991; 32:2118-2123.
2. Tjuvajev J, Muraki A, Ginos J, Berk J, Koutcher J, Ballon D, Beattie B, Finn R, Dahighian F, Blasberg R. Iododeoxyuridine uptake and retention as a measure of tumor growth. J Nucl Med. 1993; 34:1152-1162.
3. Strauss L G, Conti P S. The application of PET in clinical oncology. J Nucl Med. 1991; 32:623-648.
4. Okada J, Yoshihawa K, Itami M, Imaseki K, Uno K, Itami J, Kuyama J, Mikata A, Arimizu N. Positron emission tomography using fluorine-18-fluorodeoxyglucose in malignant lymphoma: A comparison with proliferative activity. J Nucl Med. 1992; 33:325-329.
5. Okada J; Yoshikawa K, Imaseki K, Minoshima S, Uno K, Itami J, Kuyama J, Maruno H, Arimizu N. The use of FDG-PET in the detection and management of malignant lymphoma: correlation of uptake with prognosis J Nucl Med. 1991; 32: 686-691.
6. Minn H, Joensuu H, Ahonen A, Klemi P. Fluorodeoxyglucose imaging: a method to assess the proliferative activity of human cancer in vivo. Comparison with DNA flow cytometry in head and neck tumors. Cancer 1988; 61:1776-1781.
7. Higashi K, Clavo A C, Wahl R L. Does FDG uptake measure proliferative activity of human cancer cells? In vitro comparison with DNA flow cytometry and tritiated thymidine uptake. J Nucl Med. 1992; 34:414-419.
8. McConathy J, Martarello L, Malveaux E J, Camp V M, Simpson N E, Simpson C P, Bowers G D, Zhang Z, Olson J J, Goodman M M. Synthesis and evaluation of 2-amino-4-[(18)F]fluoro-2-methylbutanoic acid (FAMB): relationship of amino acid transport to tumor imaging properties of branched fluorinated amino acids. Nucl Med Biol. 2003; 30:477-490.
9. Jager P L, Plaat B E, de Vries E G, Molenaar W M, Vaalburg W, Piers D A, Hoekstra H J. Imaging of soft-tissue tumors using L-3-[iodine-123]iodo-alpha-methyl-tyrosine single photon emission computed tomography: comparison with proliferative and mitotic activity, cellularity, and vascularity. Clin Cancer Res. 2000; 6:2252-2259.
10. Coleman R E. Single photon emission computed tomography and positron emission tomography in cancer imaging. Cancer. 1991; 67:1261-1270.
11. Kubota K, Matsuzawa T, Ito M, Ito K, Fujiwara T, Abe Y, Yoshioka S, Fukuda H, Hatazawa J, Iwata R. Lung tumor imaging by positron emission tomography using C-11 L-methionine. J Nucl Med. 1985; 26:37-42.
12. Willemsen A T M, Waarde A V, Paans A M J, Pruim J, Luurtsema G, Go K G, Vaalburg W. In vivo protein synthesis rate determination in primary or recurrent brain tumor using L-[1-¹¹C]-tyrosine and PET. J Nucl Med. 1995; 36: 411-419.
13. Tjuvajev J G, Doubrovin M, Akhurst T, Cai S, Balatoni J, Alauddin M M, Finn R, Bornmann W, Thaler H, Conti P S, Blasberg R G. Comparison of radiolabeled nucleoside probes (FIAU, FHBG, and FHPG) for PET imaging of HSV1-tk gene expression. J Nucl Med. 2002; 43:1072-1083.
14. Gambhir S S, Barrio J R, Wu L, et al. Imaging of adenoviral-directed herpes simplex virus type 1 thymidine kinase reporter gene expression in mice with radiolabeled ganciclovir. J Nucl Med. 1998; 39:2003-2011.
15. Gambhir S S, Barrio J R, Phelps M E, et al. Imaging adenoviral-directed reporter gene expression in living animals with positron emission tomography. Proc Natl Acad Sci USA. 1999; 96:2333-2338.
16. Namavari M, Barrio J R, Toyokuni T, et al. Synthesis of 8-[¹⁸F]fluoroguanine derivatives: in vivo probes for imaging gene expression with positron emission tomography. Nucl Med Biol. 2000; 27:157-162.
17. Gambhir S S, Bauer E, Black M E, et al. A mutant herpes simplex virus type 1 thymidine kinase reporter gene shows improved sensitivity for imaging reporter gene expression with positron emission tomography. Proc Natl Acad Sci USA. 2000; 97:2785-2790.
18. Iyer M, Barrio J R, Namavari M, et al. 8-[¹⁸F]Fluoropenciclovir: an improved reporter probe for imaging HSV1-tk reporter gene expression in vivo using PET. J Nucl Med. 2001; 42:96-105.
19. Alauddin M M, Conti P S, Mazza S M, et al. 9-[(3-[¹⁸F]-Fluoro-1-hydroxy-2-propoxy)methyl]guanine ([¹⁸F]-FHPG): a potential imaging agent of viral infection and gene therapy using PET. Nucl Med Biol. 1996; 23:787-792.
20. Alauddin M M, Shahinian A, Kundu R K, et al. Evaluation of 9-[(3-¹⁸F-fluoro-1-hydroxy-2-propoxy)methyl]guanine ([¹⁸F]-FHPG) in vitro and in vivo as a probe for PET imaging of gene incorporation and expression in tumors. Nucl Med Biol. 1999; 26:371-376.
21. Alauddin M M, Conti P S. Synthesis and preliminary evaluation of 9-(4-[¹⁸F]-fluoro-3-hydroxymethylbutyl)guanine ([¹⁸F]FHBG): a new potential imaging agent for viral infection and gene therapy using PET. Nucl Med Biol. 1998; 25:175-180.
22. Yaghoubi S, Barrio J R, Dahlbom M, et al. Human pharmacokinetic and dosimetry studies of [¹⁸F]FHBG: a reporter probe for imaging herpes simplex virus type-1 thymidine kinase reporter gene expression. J Nucl Med. 2001; 42:1225-1234.
23. Buck A K, Halter G, Schirrmeister H, Kotzerke J, Wurziger I, Glatting G, Mattfeldt T, Neumaier B, Reske S N, Hetzel M. Imaging Proliferation in Lung Tumors with PET: ¹⁸F-FLT Versus ¹⁸F-FDG. J Nucl Med. 2003; 44:1426-1431.
24. Francis D L, Visvikis D, Costa D C, Arulampalam T H, Townsend C, Luthra S K, Taylor I, Ell P J. Potential impact of [18F]3′-deoxy-3′-fluorothymidine versus [18F]fluoro-2-deoxy-D-glucose in positron emission tomography for colorectal cancer. Eur J Nucl Med Mol Imaging. 2003; 30:988-994.
25. Marquez V E, Tseng C K H, Mitsuya H, Aoki S, Kelly J A, Ford H, Roth J S, Broder S, Johns D G, Driscoll J S. Acid-stable 2′-fluoro purine dideoxynucleosides as active agents against HIV. J Med Chem. 1990; 33:978-985.
26. Wright J A, Taylor N F, Fox J J. Nucleosides. L X. Fluorocarbohydrates. synthesis of 2-deoxy-2-fluoro-d-arabinose and 9-(2-deoxy-2-fluoro-a-and-b-d-arabinofuranosyl)adenines. J Org Chem. 1969; 34:2632-2636.
27. Uesugi S, Kaneyasu T, Ikehara M. Synthesis and properties of ApU Analogues containing 2′-halo-2′-deoxyadenosine. Effect of 2′ substituents on oligonucleotide conformation. Biochemistry 1982; 21:5870-5877.
28. Ikehara M, Miki H. Studies of nucleosides and nucleotides. Cyclonucleosides. Synthesis and properties of 2′-halogeno-2′-deoxyadenosines. Chem. Pharm. Bull. 1978; 26: 2449-2453.
29. Malspeis L, Grever M R, Staubus A E, Young D. Pharmacokinetics of 9-B-D-arabinofuranosyl-2-fluoroadenine in cancer patients during the phase I clinical investigation of fludarabine phosphate. Seminars in Oncology 1990; 17:18-32.
30. Linn S C, Pinedo H M, van Ark-Otte J, van der Valk P, Hoekman K, Honkoop A H, Vermorken J B, Giaccone G. Expression of drug resistance proteins in breast cancer, in relation to chemotherapy. Int J Cancer. 1997; 71(5):787-795.
31. Li L, Luan Y, Wang G, Tang B, Li D, Zhang W, Li X, Zhao J, Ding H, Reed E, Li Q Q. Development and characterization of five cell models for chemoresistance studies of human ovarian carcinoma. Int J Mol Med. 2004; 14(2):257-264.
32. Long N M, Smith C S. Causes and imaging features of false positives and false negatives on F-PET/CT in oncologic imaging. Insights Into Imaging. 2011; 2(6): 679-698. doi:10.1007/s13244-010-0062-3.
33. Woolf D K, Beresford M, Li S P, et al. Evaluation of FLT-PET-CT as an imaging biomarker of proliferation in primary breast cancer. Br J Cancer. 2014; 110(12):2847-2854. doi:10.1038/bjc.2014.207.
34. Clunie G P R, Lennard L. Relevance of thiopurine methyltransferase status in rheumatology patients receiving azathioprine. Rheumatology 2004; 43(1):13-18. doi: 10.1093/rheumatology/keg442.
35. Osterman M T. Advances in IBD: Current developments in the treatment of inflammatory bowel diseases. Gastroenterol Hepatol (NY). 2009; 5(12):830-833.
36. Asadov C, Aliyeva G, Mustafayeva K. Thiopurine S-methyltransferase as a pharmacogenetic biomarker: significance of testing and review of major methods. Cardiovascular & Hematological Agents in Medicinal Chemistry. 2017; 15:23-30.

What is claimed is:

1. A compound for quantifying purine pathway-directed systems, which has the following formula: 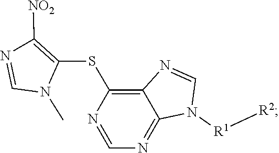
wherein R¹ is an alkyl group containing 2 to 7 carbon atoms, and one of the carbon atoms is optionally substituted by a hydroxy group; R² is a chelator with a nitrogen containing tetraazacyclic ring.

2. The compound according to claim 1, wherein the chelator is cyclam, cyclen, cyclam-carboxylic acid, or cyclen-carboxylic acid.

3. The compound according to claim 1, wherein the chelator is chelating a metal ion.

4. The compound according to claim 3, wherein the metal ion is a radionuclide, a non-radioactive metal, or a combination thereof.

5. The compound according to claim 4, wherein the radionuclide is ^{99m}Tc, ^{67,68}Ga, ^{60,61,62,64,67}Cu, ¹¹¹In, ¹⁶⁶Ho, ^{186,188}Re, ⁹⁰Y, ¹⁷⁷Lu, ²²³Ra, ²²⁵Ac, and ⁸⁹Zr, ^{117m}Sn, ¹⁵³Sm, ⁸⁹Sr, ⁵⁹Fe, ²¹²Bi, ²¹¹At, ⁴⁵Ti, or a combination thereof.

6. The compound according to claim 5, wherein the non-radioactive metal is Tc, Sn, Cu, In, Tl, Ga, As, Re, Ho, Y, Sm, Se, Sr, Gd, Bi, Fe, Mn, Lu, Co, Pt, Ca, Rh, Eu, Tb, or a combination thereof.

7. The compound according to claim 1, which has one of the following formulas:

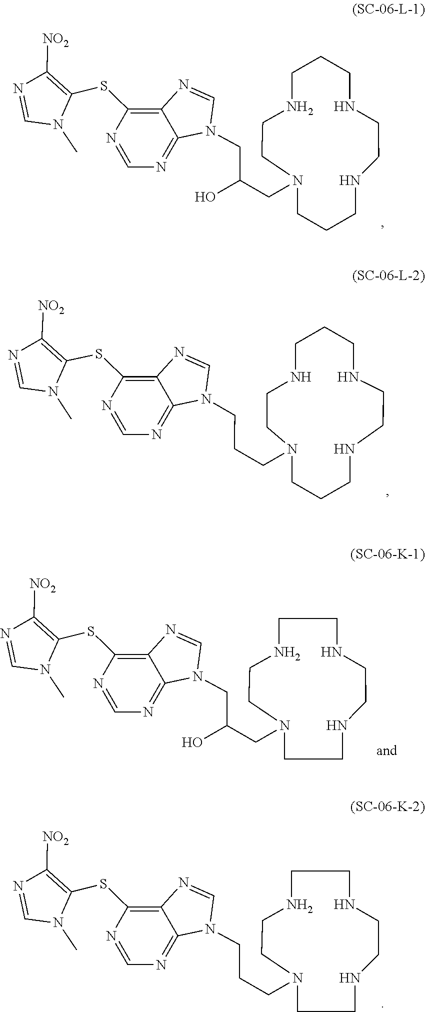

8. A kit comprising the compound according to claim 1 and an instrument for administering the pharmaceutical composition.

9. A method of preparing the compound according to claim 1, comprising:
reacting a compound represented by Chemical Formula 1 with a compound represented by Chemical Formula 2

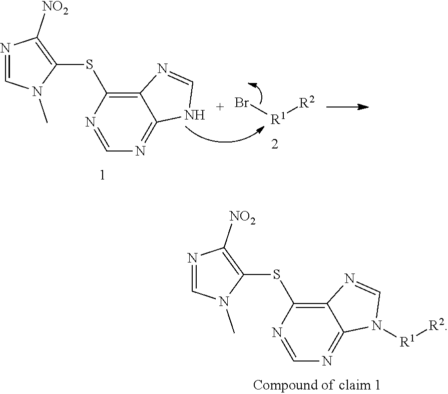

10. A method of scanning tumors, which comprise administering to an animal containing tumors an imaging amount of the compound according to claim 1, and imaging the bones with an imaging technology for detecting cancer, autoimmune disorder (multiple sclerosis, myasthenia gravis, rheumatoid arthritis, systemic lupus erythematosus/Lupus nephritis, inflammatory bowel disease, ulcerative colitis, idiopathic pulmonary fibrosis, hepatitis), bone marrow disease, atherosclerosis.

11. The method according to claim 10, wherein the image technology is CT, MRI, PET and/or SPECT.

12. The method according to claim 11, wherein the imaging amount is defined as a kit.

13. A treatment method for cancer, anti-metabolite, anti-proliferation (S/G1) autoimmune disorder (multiple sclerosis, myasthenia gravis, rheumatoid arthritis, systemic lupus erythematosus/Lupus nephritis, inflammatory bowel disease, ulcerative colitis, idiopathic pulmonary fibrosis, hepatitis), bone marrow disease, comprising administration of the compound according to claim 1.
